(19) United States
(12) Patent Application Publication
(10) Pub. No.: US 2007/0060584 A1
(43) Pub. Date: Mar. 15, 2007

(54) Biaryl substituted thiazoles, oxazoles and imidazoles as sodium channel blockers

(76) Inventors: Prasun K. Chakravarty, Edison, NJ (US); Michael H. Fisher, Ringoes, NJ (US); William H. Parsons, Belle Mead, NJ (US); Sriram Tyagarajan, Edison, NJ (US); Bishan Zhou, Edison, NJ (US); Jeffrey M. Fisher, Ringoes, NJ (US)

Correspondence Address: MERCK AND CO., INC, P O BOX 2000, RAHWAY, NJ, 07065-0907, US

(21) Appl. No.: 10/553,554

(22) PCT Filed: Apr. 14, 2004
(86) PCT No.: PCT/US04/11271

§ 371 Date: Oct. 18, 2005

Related U.S. Application Data
(60) Provisional application No. 60/444,523, filed on Jan. 31, 2003.

(51) Int. Cl. A61K 31/5377; A61K 31/497; A61K 31/496; A61K 31/454; A61K 31/427; A61K 31/422; A61K 31/4178; C07D 413/02; C07D 417/02; C07D 403/02

(52) U.S. Cl. 514/235.2; 514/235.5; 514/254.02; 514/254.05; 514/255.05; 514/256; 514/326; 514/365; 514/374; 514/397; 544/135; 544/139; 544/140; 544/405; 544/333; 544/369; 544/370; 544/371; 546/209; 546/210; 546/211; 548/152; 548/215; 548/311.1

(57) ABSTRACT

Biaryl substituted thiazole, oxazole and imidazole compounds are sodium channel blockers useful for the treatment of pain. Pharmaceutical compositions comprise an effective amount of the instant compounds, either alone, or in combination with one or more therapeutically active compounds, and a pharmaceutically acceptable carrier. Methods of treatment or prevention of conditions, including acute pain, chronic pain, visceral pain, inflammatory pain, and neuropathic pain comprise administering an effective amount of the present compounds, either alone, or in combination with one or more therapeutically active compounds.

### FIELD OF THE INVENTION

The present invention is directed to a series of biaryl substituted thiazole, oxazole and imidazole compounds. In particular, this invention is directed to biaryl substituted thiazole, oxazole and imidazole compounds that are sodium channel blockers useful for the treatment of chronic and neuropathic pain. The compounds of the present invention are also useful for the treatment of other conditions, including, for example, central nervous system (CNS) disorders such as anxiety, depression, epilepsy, manic depression, bipolar disorder and diabetic neuropathy.

### BACKGROUND OF THE INVENTION

Voltage-gated ion channels allow electrically excitable cells to generate and propagate action potentials and therefore are crucial for nerve and muscle function. Sodium channels play a special role by mediating rapid depolarization, which constitutes the rising phase of the action potential and in turn activates voltage-gated calcium and potassium channels. Voltage-gated sodium channels represent a multigene family. Nine sodium channel subtypes have been cloned and functionally expressed to date. [Clare, J. J., Tate, S. N., Nobbs, M. & Romanos, M. A. Voltage-gated sodium channels as therapeutic targets. Drug Discovery Today 5, 506-520 (2000)]. They are differentially expressed throughout muscle and nerve tissues and show distinct biophysical properties. All voltage-gated sodium channels are characterized by a high degree of selectivity for sodium over other ions and by their voltage-dependent gating. [Catterall, W. A. Structure and function of voltage-gated sodium and calcium channels. Current Opinion in Neurobiology 1, 5-13 (1991)]. At negative or hyperpolarized membrane potentials, sodium channels are closed. Following membrane depolarization, sodium channels open rapidly and then inactivate. Sodium channels only conduct currents in the open state and, once inactivated, have to return to the resting state, favored by membrane hyperpolarization, before they can reopen. Different sodium channel subtypes vary in the voltage range over which they activate and inactivate as well as in their activation and inactivation kinetics.

Sodium channels are the target of a diverse array of pharmacological agents, including neurotoxins, antiarrhythmics, anticonvulsants and local anesthetics. [Clare, J. J., Tate, S. N., Nobbs, M. & Romanos, M. A. Voltage-gated sodium channels as therapeutic targets. Drug Discovery Today 5, 506-520 (2000)]. Several regions in the sodium channel secondary structure are involved in interactions with these blockers and most are highly conserved. Indeed, most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrigine, phenytoin and carbamazepine) and certain cardiac arrhythmias (e.g. lignocaine, tocainide and mexiletine).

It is well known that the voltage-gated Na⁺ channels in nerves play a critical role in neuropathic pain. Injuries of the peripheral nervous system often result in neuropathic pain persisting long after the initial injury resolves. Examples of neuropathic pain include, but are not limited to, postherpetic neuralgia, trigeminal neuralgia, diabetic neuropathy, chronic lower back pain, phantom limb pain, pain resulting from cancer and chemotherapy, chronic pelvic pain, complex regional pain syndrome and related neuralgias. It has been shown in human patients as well as in animal models of neuropathic pain, that damage to primary afferent sensory neurons can lead to neuroma formation and spontaneous activity, as well as evoked activity in response to normally innocuous stimuli. [Carter, G. T. and B. S. Galer, Advances in the management of neuropathic pain. Physical Medicine and Rehabilitation Clinics of North America, 2001. 12(2): p. 447-459]. The ectopic activity of normally silent sensory neurons is thought to contribute to the generation and maintenance of neuropathic pain. Neuropathic pain is generally assumed to be associated with an increase in sodium channel activity in the injured nerve. [Baker, M. D. and J. N. Wood, Involvement of Na channels in pain pathways. TRENDS in Pharmacological Sciences, 2001. 22(1): p. 27-31].

Indeed, in rat models of peripheral nerve injury, ectopic activity in the injured nerve corresponds to the behavioral signs of pain. In these models, intravenous application of the sodium channel blocker and local anesthetic lidocaine can suppress the ectopic activity and reverse the tactile allodynia at concentrations that do not affect general behavior and motor function. [Mao, J. and L. L. Chen, Systemic lidocaine neuropathic pain relief. Pain, 2000. 87: p. 7-17]. These effective concentrations were similar to concentrations shown to be clinically efficacious in humans. [Tanelian, D. L. and W. G. Brose, Neuropathic pain can be relieved by drugs that are use-dependent sodium channel blockers: lidocaine, carbamazepine and mexiletine. Anesthesiology, 1991. 74(5): p. 949-951]. In a placebo-controlled study, continuous infusion of lidocaine caused reduced pain scores in patients with peripheral nerve injury, and in a separate study, intravenous lidocaine reduced pain intensity associated with postherpetic neuralgia (PHN). [Mao, J. and L. L. Chen, Systemic lidocaine for neuropathic pain relief. Pain, 2000. 87: p. 7-17. Anger, T., et al., Medicinal chemistry of neuronal voltage-gated sodium channel blockers. Journal of Medicinal Chemistry, 2001. 44(2): p. 115-137]. Lidoderm®, lidocaine applied in the form of a dermal patch, is currently the only FDA approved treatment for PHN. [Devers, A. and B. S. Galer, Topical lidocaine patch relieves a variety of neuropathic pain conditions: an open-label study. Clinical Journal of Pain, 2000. 16(3): p. 205-208].

In addition to neuropathic pain, sodium channel blockers have clinical uses in the treatment of epilepsy and cardiac arrhythmias. Recent evidence from animal models suggests that sodium channel blockers may also be useful for neuroprotection under ischaemic conditions caused by stroke or neural trauma and in patients with multiple sclerosis (MS). [Clare, J. J. et. al And Anger, T. et al.].

International Patent Publication WO 00/57877 describes aryl substituted imidazoles, oxazoles, thiazoles, and pyrroles and their uses as sodium channel blockers. International Patent Publication WO 01/68612 describes aryl substituted pyridines, pyrimidines, pyrazines and triazines and their uses as sodium channel blockers. International Patent Publication WO 99/32462 describes triazine compounds for the treatment for CNS disorders. However, there remains a need for novel compounds and compositions that therapeutically block neuronal sodium channels with less side effects and higher potency than currently known compounds.

### SUMMARY OF THE INVENTION

The present invention is directed to biaryl thiazoles, oxazoles and imidazoles that are sodium channel blockers useful for the treatment of chronic and neuropathic pain. The compounds of the present invention are also useful for the treatment of other conditions, including CNS disorders such as depression, anxiety, epilepsy, manic depression and bipolar disorder. This invention provides pharmaceutical compositions comprising a compound of the present invention, either alone, or in combination with one or more therapeutically active compounds, and a pharmaceutically acceptable carrier.

This invention further comprises methods for the treatment of conditions associated with, or resulting from, sodium channel activity, such as acute pain, chronic pain, visceral pain, inflammatory pain, neuropathic pain and disorders of the CNS including, but not limited to, epilepsy, manic depression and bipolar disorder.

### DETAILED DESCRIPTION OF THE INVENTION

The compounds described in the present invention are represented by Formula (I): 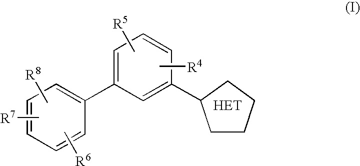
or pharmaceutically acceptable salts thereof, wherein

HET is one of the following heterocycles: 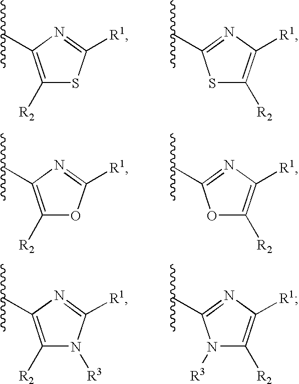
R¹ is
(a) H;
(b) C₁-C₆-alkyl, C₂-C₄-alkenyl, C₂-C₄-alkynyl, C₃-C₆-cycloalkyl, or C₁-C₄alkyl-[C₃-C₆-cycloalkyl], any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —O—C₁-C₆-alkyl, —O—C₃-C₆-cycloalkyl, —S—C₁-C₆-alkyl or —S—C₃-C₆-cycloalkyl, any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isoxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(d) —C₀-C₄-alkyl-C₁-C₄-perfluoroalkyl, or —O—C₀-C₄-alkyl-C₁-C₄-perfluoroalkyl;
(e) —OH;
(f) —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(g) —OCON(R^{a})(R^{b}), or —OSO₂N(R^{a})(R^{b});
(h) —SH, or —SCON(R^{a})(R^{b});
(i) NO₂;
(j) NR^{a}R^{b}, —N(COR^{a})R^{b}, —N(SO₂R^{a})R^{b}, —N(R^{a})SO₂N(R^{b})₂, —N(OR^{a})CONR^{a}R^{b}, —N(R^{a})SO₂R^{a} or —N(R^{a})CON(R^{a})₂;
(k) —CH(OR^{a})R^{a}, —C(OR^{b})CF₃, —CH(NHR^{b})R^{a}, —C(═O)R^{a}, C(═O)CF₃, —SOCH₃, —SO₂CH₃, COOR^{a}, CN, CONR^{a}R^{b}, —COCONR^{a}R^{b}, —SO₂NR^{a}R^{b}, —CH₂O—SO₂NR^{a}R^{b}, SO₂N(R^{a})OR^{a}, —C(═NH)NH₂, —CR^{a}═N—OR^{a}, CH═CHCONR^{a}R^{b};
(l) —CONR^{a}(CH₂)₀₋₂C(R^{a})(R^{b})(CH₂)₀₋₂CONR^{a}R^{b};
(m) tetrazolyl, tetrazolinonyl, triazolyl, triazolinonyl, imidazolyl, imidozolonyl, oxazolyl, oxadiazolyl, isooxazolyl, thiazolyl, furyl, thienyl, pyrazolyl, pyrazolonyl, pyrrolyl, pyridyl, pyrimidinyl, pyrazinyl, or phenyl, any of which is optionally substituted with 1-3 substituents selected from i) F, Cl, Br, I, ii) —CN, iii) —NO₂, iv) —C(═O)R^{a}, v) C₁-C₆-alkyl, vi) —O—R^{a}, vii) —NR^{a}R^{b}, viii) —C₀-C₄-alkyl-CO—OR^{a}, ix) —(C₀-C₄-alkyl)—NH—CO—OR^{a}, x) —(C₀-C₄-alkyl)—CO—NR^{a}R^{b}, xi) —S(O)₀₋₂R^{a}, xii) —SO₂NR^{a}R^{b}, xiii) —NHSO₂R^{a}, xiv) —C₁-C₄-perfluoroalkyl, and xv) —O—C₁-C₄-perfluoroalkyl;
(n) —C(R^{a})═C(R^{b})—COOR^{a}, or —C(R^{a})═C(R^{b})—CONR^{a}R^{b};
(o) 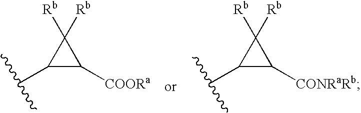
(p) piperidin-1-yl, morpholin-4-yl, pyrrolidin-1-yl, piperazin-1-yl or 4-substituted piperazin-1-yl, any of which is optionally substituted with 1-3 substituents selected from i) —CN, ii) —C(═O)(R^{a}), iii) C₁-C₆-alkyl, iv) —OR^{a}, v) —NR^{a}R^{b}, vi) —C₀-C₄-alkyl-CO—OR^{a}, vii) —(C₀-C₄-alkyl)-NH—CO—OR^{a}, viii) —(C₀-C₄-alkyl)—CON(R^{a})(R^{b}), ix) —SR^{a}, x) —S(O)₀₋₂R^{a}, xi) —SO₂N(R^{a})(R^{b}), xii) —NR^{a}SO₂R^{a} xiii) —C₁-C₄-perfluoroalkyl and xiv) —O—C₁-C₄-perfluoroalkyl;
   R^{a} is
(a) H;
(b) C₁-C₄-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), —OCON(C₁-C₄alkyl)(C₁-C₄alkyl), —OCONHC₁-C₄alkyl-aryl), —OCON(C₁-C₄alkyl)(C₁-C₄alkyl-aryl), NH₂, NH(C₁-C₄alkyl), N(C₁-C₄alkyl)(C₁-C₄alkyl), NH(C₁-C₄alkyl-aryl), N(C₁-C₄alkyl)(C₁-C₄alkyl-aryl), NHCONH₂, NHCONH(C₁-C₄alkyl), NHCONH(C₁-C₄alkyl-aryl), —NHCON(C₁-C₄alkyl)(C₁-C₄alkyl), NHCON(C₁-C₄alkyl)(C₁-C₄alkyl-aryl), N(C₁-C₄alkyl)CON(C₁-C₄alkyl)(C₁-C₄alkyl), N(C₁-C₄alkyl)CON(C₁-C₄alkyl)(C₁-C₄alkyl-aryl), COO—(C₁-C₄-alkyl), COOH, CN, CONH₂, CONH(C₁-C₄alkyl), CON(C₁-C₄alkyl)(C₁-C₄alkyl), SO₂NH₂, SO₂NH(C₁-C₄alkyl), SO₂NH(C₁-C₄alkyl-aryl), SO₂N(C₁-C₄alkyl)(C₁-C₄alkyl), NHSO₂, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) C₀-C₄-alkyl-(C₁-C₄)-perfluoroalkyl; or
(d) —C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(C₁-C₄-alkyl), v) —O(C₁-C₄-alkyl), vi) —N(C₁-C₄-alkyl)(C₁-C₄-alkyl), vii) —C₁₋₁₀alkyl, and viii) —C₁₋₁₀alkyl, wherein one or more of the alkyl carbons can be replaced by a —O—, —S(O)₁₋₂—, —O—C(O)—, —C(O)—O—, —C(O)—, —CH(OH)—, —C═C—, or —C≡C—;
   R^{b} is
(a) H; or
(b) C₁-C₆-alkyl, optionally substituted with one or more of the following substituents: F, CF₃, OH, O—(C₁-C₄)allyl, S(O)₀₋₂—(C₁-C₄)alkyl, —OCONH₂, —OCONH(C₁-C₄alkyl), NH₂, NH(C₁-C₄alkyl), N(C₁-C₄alkyl)(C₁-C₄alkyl), NHCONH₂, NHCONH(C₁-C₄alkyl), —NHCON(C₁-C₄alkyl)(C₁-C₄alkyl), COO—(C₁-C₄-alkyl), COOH, CN, or CONH₂;
   R² is:
(a) H;
(b) —C₁-C₄-alkyl, —C₃-C₆-cycloalkyl or —C₁-C₄-alkyl-(C₃-C₆)-cycloalkyl, optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —C₀-C₄-alkyl-C₁-C₄-perfluoroalkyl;
(d) aryl or —(C₁-C₄-alkyl)-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(e) —C(═O)(R^{a}), —CONR^{a}R^{b}, COO—(C₁-C₄)alkyl, —SO₂R^{a}, —SO₂N(R^{a})(R^{b});
   R³ is
(a) H;
(b) —C₁-C₄-alkyl, —C₃-C₆-cycloalkyl or —C₁-C₄-alkyl-(C₃-C₆)-cycloalkyl, optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —C₀-C₄-alkyl-C₁-C₄-perfluoroalkyl;
(d) aryl or —(C₁-C₄-alkyl)-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—,
(e) —O-C₁-C₄-alkyl, —O—C₀-C₄-alkyl-C₁-C₄-perfluoroalkyl, —O-aryl or —O(C₁-C₄-alkyl)-aryl;
(f) —C(═O)(R^{a}), —SO₂R^{a}, —SO₂N(R^{a})(R^{b}), CN, NR^{a}R^{b}, NO₂, F, Cl, Br, I, OH, OCONR^{a}R^{b}, O(C₁-C₄-alkyl)CONR^{a}R^{b}, —OSO₂NR^{a}R^{b}, COOR^{a}, or CONR^{a}R^{b};
   R⁴ and R⁵ each independently is:
(a) H;
(b) —C₁-C₆-alkyl, —C₂-C₆-alkenyl, —C₂-C₆-alkynyl or —C₃-C₆-cycloalkyl, any of which is optionally substituted with one or more of the following substituents: F, CF₃, —O—(C₁-C₄)alkyl, CN, —N(R^{a})(R^{b}), —N(R^{a})CO—(C₁-C₄)alkyl, COOR^{b}, CON(R^{a})(R^{b}) or phenyl;
(c) —O—C₀-C₆-alkyl, —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂, —O—C(O)—, —C(O)—O—, —C(O)—N(R^{a})—,
   —N(R^{a})—C(O)—, —N(R^{a})—C(O)—N(R^{a})—, —C(O)—, —CH(OH)—, —C═C—, or —C≡C—;
(d) —C₀-C₄-alkyl-C₁-C₄-perfluoroalkyl, or —O—C₀-C₄-alkyl-C₁-C₄-perfluoroalkyl; or
(e) CN, NH₂, NO₂, F, Cl, Br, I, OH, OCON(R^{a})(R^{b})O(C₁-C₄-alkyl)CONR^{a}R^{b}, —OSO₂N(R^{a})(R^{b}), COOR^{b}, CON(R^{a})(R^{b}), or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; and
   R⁶, R⁷ and R⁸ each independently is:
(a) H;
(b) C₁-C₆-alkyl, C₂-C₄-alkenyl, C₂-C₄-alkynyl or C₃-C₆-cycloalkyl, any of which is optionally substituted with one or more of the following substituents: F, CF₃, OH, O—(C₁-C₄)alkyl, OCON(R^{a})(R^{b}), NR^{a}R^{b}, COOR^{a}, CN, CONR^{a}R^{b}, N(R^{a})CONR^{a}R^{b}, N(R^{a})SO₂NR^{a}R^{b}, SO₂NR^{a}R^{b}, S(O)₀₋₂(C₁-C₄-alkyl), —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(c) —O—C₁-C₆-alkyl, —O—C₃-C₆-cycloalkyl, —S—C₁-C₆-alkyl or —S—C₃-C₆-cycloalkyl, any of which is optionally substituted with one or more of the following substituents: F, CF₃, OH, O—(C₁-C₄)alkyl, NH₂, NH(C₁-C₄-alkyl), N(C₁-C₄-alkyl)₂, COOH, CN, CONH₂, CONH(C₁-C₄-alkyl), CONH(C₁-C₄-alkyl)₂, SO₂NH₂, SO₂NH(C₁-C₄-alkyl), tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(d) —C₀-C₄-alkyl-C₁-C₄-perfluoroalkyl, or —O—C₀-C₄-alkyl-C₁-C₄-perfluoroalkyl;
(e) —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; (f) CN, N(R^{a})(R^{b}), NO₂, F, Cl, Br, I, —OR^{a}, —SR^{a}, —OCON(R^{a})(R^{b}), —OSO₂N(R^{a})(R^{b}), COOR^{b}, CON(R^{a})(R^{b}), —N(R^{a})CON(R^{a})(R^{b}), —N(R^{a})SO₂N(R^{a})(R^{b}), —C(OR^{b})R^{a}, —C(OR^{a})CF₃, —C(NHR^{a})CF₃, —C(═O)R^{a}, C(═O)CF₃, —SOCH₃, —SO₂CH₃, —NHSO₂(C₁₋₆-alkyl), —NHSO₂-aryl, SO₂N(R^{a})(R^{b}), —CH₂OSO₂N(R^{a})(R^{b}), SO₂N(R^{b})—OR^{a}, —C(═NH)NH₂, —CRₐ═N—ORₐ, CH═CH or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C≡C—, or —C≡C; or when R⁶ and R7 are present on adjacent carbon atoms, R6 and R7, together with the benzene ring to which they are attached, may form a bicyclic aromatic ring selected from naphthyl, indolyl, quinolinyl, isoquinolinyl, quinoxalinyl. benzofuryl, benzothienyl, benzoxazolyl, benzothiazolyl, and benzimidazolyl, any aromatic ring of which is optionally substituted with 1-4 independent substituents selected from i) halogen, ii) —CN, iii) —NO₂, iv) —CHO, v) —O—C₁₋₄alkyl, vi) —N(C₀₋₄alkyl)(C₀₋₄alkyl), vii) —C₀₋₄alkyl-CO—O(C₀₋₄alkyl), viii) —(C₀₋₄alkyl)-NH—CO—O(C₀₋₄alkyl), ix) —(C₀₋₄alkyl)—CO—N(C₀₋₁alkyl)(C₀₋₄alkyl), x) —S(C₀₋₄alkyl), xi) —S(O)(C₁₋₄alkyl), xii) —SO₂(C₀₋₄alkyl), xiii) —SO₂N(C₀₋₄alkyl)(C₀₋₄alkyl), xiv) —NHSO₂(C_{0 -4}alkyl)(C₀₋₄alkyl), xv) —C₁₋₁₀alkyl and xvi) —C₁₋₁₀alkyl in which one or more of the carbons can be replaced by a —N(C₀₋₆alkyl)-, —O—, —S(O)₁₋₂—, —O—C(O)—, —C(O)—O—, —C(O)—N(C₀₋₆alkyl)-, —N(C_{0 -6}alkyl)—C(O)—, —N(C₀₋₆alkyl)-C(O)—N(C₀₋₆alkyl)-, —C(O)—, —CH(OH), —C═C—, or —C≡C—.

In one aspect, the present invention provides a compound described by the chemical Formula (I), or a pharmaceutically acceptable salt thereof, wherein

R⁶ is other than H and is attached at the ortho position.

In a second aspect, the present invention provides a compound described by the chemical Formula (I), or a pharmaceutically acceptable salt thereof, wherein

HET is

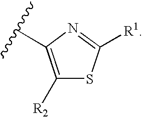

In a third aspect, the present invention provides a compound described by the chemical Formula (I), or a pharmaceutically acceptable salt thereof, wherein

HET is

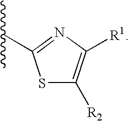

In a fourth aspect, the present invention provides a compound described by the chemical Formula (I), or a pharmaceutically acceptable salt thereof, wherein

HET is

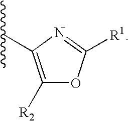

In a fifth aspect, the present invention provides a compound described by the chemical Formula (I), or a pharmaceutically acceptable salt thereof, wherein

HET is

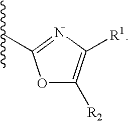

In a sixth aspect, the present invention provides a compound described by the chemical Formula (I), or a pharmaceutically acceptable salt thereof, wherein

HET is

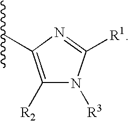

In a seventh aspect, the present invention provides a compound described by the chemical Formula (I), or a pharmaceutically acceptable salt thereof, wherein

HET is

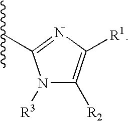

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, and 1,2,3,4-tetrahydronaphalene. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, and indenyl.

The term “aryl” includes, but is not limited to, an aromatic substituent that is a single ring or multiple rings fused together. When formed of multiple rings, at least one of the constituent rings is aromatic. The term “aryl”, unless specifically noted otherwise, also includes heteroaryls, and thus includes stable 5- to 7-membered monocyclic and stable 9- to 10-membered fused bicyclic heterocyclic ring systems that consist of carbon atoms and from one to four heteroatoms selected from the group consisting of N, O and S, wherein the nitrogen and sulfur heteroatoms may optionally be oxidized, and the nitrogen heteroatom may optionally be quaternized. Suitable aryl groups include phenyl, naphthyl, pyridyl, pyrimidinyl, furyl, thienyl, pyrrolyl, triazolyl, pyrazolyl, thiazolyl, isoxazolyl, oxazolyl, and oxadiazolyl.

The term “cycloalkyloxy,” unless specifically stated otherwise, includes a cycloalkyl group connected by a short length C₁₋₂alkyl to the oxy connecting atom.

The term “C₀₋₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group.

The term “hetero,” unless specifically stated otherwise, includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear, for an O or S heteroatom.

The term “amine,” unless specifically stated otherwise, includes primary, secondary and tertiary amines.

The term “carbonyl,” unless specifically stated otherwise, includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the alkyl and the aryl groups are optionally substituted. If only one of the multiple moieties is optionally substituted then it will be specifically recited such as “an alkylaryl, the aryl optionally substituted with halogen or hydroxyl.”

Compounds described herein may contain one or more double bonds and may thus give rise to cis/trans isomers as well as other conformational isomers. The present invention includes all such possible isomers as well as mixtures of such isomers unless specifically stated otherwise.

Compounds described herein can contain one or more asymmetric centers and may thus give rise to diastereoisomers and optical isomers. The present invention includes all such possible diastereoisomers as well as their racemic mixtures, their substantially pure resolved enantiomers, all possible geometric isomers, and pharmaceutically acceptable salts thereof. The above chemical Formula is shown without a definitive stereochemistry at certain positions. The present invention includes all stereoisomers of the chemical Formula and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The pharmaceutical compositions of the present invention comprise a compound represented by Formula I (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) selective serotonin reuptake inhibitors (“SSRI”) and/or selective serotonin and norepinephrine reuptake inhibitors (“SSNRI”), x) tricyclic antidepressant drugs, xi) norepinephrine modulators, xii) lithium, xiii) valproate, and xiv) neurontin (gabapentin). The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics. Compounds of this invention are useful for the treatment of irritable bowel syndrome and related disorders, as well as Crohn's disease.

The instant compounds have clinical uses for the treatment of epilepsy and partial and generalized tonic seizures. They are also useful for neuroprotection under ischaemic conditions caused by stroke or neural trauma and for treating multiple sclerosis. The present compounds are useful for the treatment of tachy-arrhythmias. Additionally, the instant compounds are useful for the treatment of neuropsychiatric disorders, including mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, neurokinin-1 receptor antagonists, corticotropin releasing factor (CRF) antagonists, and pharmaceutically acceptable salts thereof.

Further, it is understood that compounds of this invention can be administered at prophylactically effective dosage levels to prevent the above-recited conditions and disorders, as well as to prevent other conditions and disorders associated with sodium channel activity.

Creams, ointments, jellies, solutions, or suspensions containing the instant compounds can be employed for topical use. Mouth washes and gargles are included within the scope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds represented by Formula I, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and. thus, should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented by Formula I, or a pharmaceutically acceptable salt thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound described by Formula I, or a pharmaceutically acceptable salt thereof, can also be prepared in powder or liquid concentrate form.

The compounds and pharmaceutical compositions of this invention have been found to block sodium channels. Accordingly, an aspect of the invention is the treatment in mammals of maladies that are amenable to amelioration through blockage of neuronal sodium channels, including, for example, acute pain, chronic pain, visceral pain, inflammatory pain, and neuropathic pain by administering an effective amount of a compound of this invention. The term “mammals” includes humans, as well as other animals, such as, for example, dogs, cats, horses, pigs, and cattle. Accordingly, it is understood that the treatment of mammals other than humans refers to the treatment of clinical conditions in non-human mammals that correlate to the above-recited conditions.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, and xiv) neurontin (gabapentin).

The abbreviations used herein have the following tabulated meanings. Abbreviations not tabulated below have their meanings as commonly used unless specifically stated otherwise.

| | |
|---|---|
| Ac | Acetyl |
| AIBN | 2,2′-azobis(isobutyronitrile) |
| BINAP | 1,1′-bi-2-naphthol |
| Bn | Benzyl |
| CAMP | cyclic adenosine-3′,5′-monophosphate |
| CDI | Carbonyldiimidazole |
| DAST | (diethylamino)sulfur trifluoride |
| DEAD | diethyl azodicarboxylate |
| DBU | 1,8-diazabicyclo[5.4.0]undec-7-ene |
| DIBAL | diisobutylaluminum hydride |
| DMAP | 4-(dimethylamino)pyridine |
| DME | Dimethoxyethane |
| DMSO | Dimethylsulfoxide |
| DMF | N,N-dimethylformamide |
| Dppf | 1,1′-bis(diphenylphosphino)-ferrocene |
| EDCI | 1-(3-dimethylaminopropyl)-3-ethylcarbodiimide |
| | hydrochloride |
| Et₃N | Triethylamine |
| GST | glutathione transferase |
| HMDS | Hexamethyldisilazide |
| HOBT | 1-Hydroxybenztriazole |
| LDA | lithium diisopropylamide |
| m-CPBA | metachloroperbenzoic acid |
| MMPP | monoperoxyphthalic acid |
| MPPM | monoperoxyphthalic acid, magnesium salt 6H₂O |
| Ms | methanesulfonyl = mesyl = SO₂Me |
| Ms0 | methanesulfonate = mesylate |
| NBS | N-bromo succinimide |
| NCS | N-chloro succinimide |
| NSAID | non-steroidal anti-inflammatory drug |
| o-Tol | ortho-tolyl |
| OXONE ® | 2KHSO₅.KHSO₄.K₂SO₄ |
| PCC | pyridinium chlorochromate |
| Pd₂(dba)₃ | Bis(dibenzylideneacetone) palladium(0) |
| PDC | pyridinium dichromate |
| PDE | Phoshodiesterase |
| Ph | Phenyl |
| Phe | Benzenediyl |
| PMB | para-methoxybenzyl |
| Pye | Pyridinediyl |
| r.t. or RT | room temperature |
| Rac. | Racemic |
| SAM | aminosulfonyl or sulfonamide or SO₂NH₂ |
| SEM | 2-(trimethylsilyl)ethoxymethoxy |
| SPA | scintillation proximity assay |
| TBAF | tetra-n-butylammonium fluoride |
| Th | 2- or 3-thienyl |
| TFA | trifluoroacetic acid |
| TFAA | trifluoroacetic acid anhydride |
| THF | Tetrahydrofuran |
| Thi | Thiophenediyl |
| TLC | thin layer Chromatography |
| TMS-CN | trimethylsilyl cyanide |
| TMSI | trimethylsilyl iodide |
| Tz | 1H (or 2H)-tetrazol-5-yl |
| XANTPHOS | 4,5-Bis-diphenylphosphanyl-9,9-dimethyl-9H-xanthene |
| C₃H₅ | Allyl |

ALKYL GROUP ABBREVIATIONS

| | |
|---|---|
| Me = | Methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

The following in vitro and in vivo assays were used in assessing the biological activity of the instant compounds.

Compound Evaluation (In Vitro Assay):

The identification of inhibitors of the sodium channel is based on the ability of sodium channels to cause cell depolarization when sodium ions permeate through agonist-modified channels. In the absence of inhibitors, exposure of an agonist-modified channel to sodium ions will cause cell depolarization. Sodium channel inhibitors will prevent cell depolarization caused by sodium ion movement through agonist-modified sodium channels. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)). Oxanol is a lipophilic anion and distributes across the membrane according to membrane potential. In the presence of a sodium channel agonist, but in the absence of sodium, the inside of the cell is negative with respect to the outside, oxanol is accumulated at the outer leaflet of the membrane and excitation of coumarin will cause FRET to occur. Addition of sodium will cause membrane depolarization leading to redistribution of oxanol to the inside of the cell, and, as a consequence, to a decrease in FRET. Thus, the ratio change (donor/acceptor) increases after membrane depolarization. In the presence of a sodium channel inhibitor, cell depolarization will not occur, and therefore the distribution of oxanol and FRET will remain unchanged.

Cells stably transfected with the PN1 sodium channel (HEK-PN1) were grown in polylysine-coated 96-well plates at a density of ca. 140,000 cells/well. The media was aspirated, and the cells were washed with PBS buffer, and incubated with 100 μL of 10 μM CC₂-DMPE in 0.02% pluronic acid. After incubation at 25° C. for 45 min, media was removed and cells were washed 2× with buffer. Cells were incubated with 100 μL of DiSBAC₂(3) in TMA buffer containing 20 μM veratridine, 20 nM brevetoxin-3, and test sample. After incubation at 25° C. for 45 min in the dark, plates were placed in the VIPR instrument, and the fluorescence emission of both CC₂-DMPE and DiSBAC₂(3) recorded for 10 s. At this point, 100 μL of saline buffer was added to the wells to determine the extent of sodium-dependent cell depolarization, and the fluorescence emission of both dyes recorded for an additional 20 s. The ratio CC₂-DMPE/DiSBAC₂(3), before addition of saline buffer equals 1. In the absence of inhibitors, the ratio after addition of saline buffer is >1.5. When the sodium channel has been completely inhibited by either a known standard or test compound, this ratio remains at 1. It is possible, therefore, to titrate the activity of a sodium channel inhibitor by monitoring the concentration-dependent change in fluorescence ratio.

Electrophysiological Assays (In Vitro Assays):

Cell preparation: A HEK-293 cell line stably expressing the PN1 sodium channel subtype was established in-house. The cells were cultured in MEM growth media (Gibco) with 0.5 mg/mL G418, 50 units/mL Pen/Strep and 1 mL heat-inactivated fetal bovine serum at 37° C. and 10% CO₂. For electrophysiological recordings, cells were plated on 35 mm dishes coated with poly-D-lysine.

Whole-cell recordings: HEK-293 cells stably expressing the PN1 sodium channel subtype were examined by whole cell voltage clamp (Hamill et. al. Pfluegers Archives 391:85-100 (1981)) using an EPC-9 amplifier and Pulse software (HEKA Electronics, Lamprecht, Germany). Experiments were performed at room temperature. Electrodes were fire-polished to resistances of 2-4 MΩ. Voltage errors were minimized by series resistance compensation, and the capacitance transient was canceled using the EPC-9's built-in circuitry. Data were acquired at 50 kHz and filtered at 7-10 kHz. The bath solution consisted of 40 mM NaCl, 120 mM NMDG Cl, 1 mM KCl, 2.7 mM CaCl₂, 0.5 mM MgCl₂, 10 mM NMDG HEPES, pH 7.4, and the internal (pipet) solution contained 110 mM Cs-methanesulfonate, 5 mM NaCl, 20 mM CsCl, 10 mM CsF, 10 mM BAPTA (tetra Cs salt), 10 mM Cs HEPES, pH7.4.

The following protocols were used to estimate the steady-state affinity of compounds for the resting and inactivated state of the channel (Kᵣ and Kᵢ, respectively):

1) 8 ms test-pulses to depolarizing voltages from −60 mV to +50 mV from a holding potential of −90 mV were used to construct current-voltage relationships (IV-curves). A voltage near the peak of the IV-curve (typically −10 or 0 mV) was used as the test-pulse voltage throughout the remainder of the experiment.

2) Steady-state inactivation (availability) curves were constructed by measuring the current activated during an 8 ms test-pulse following 10 s conditioning pulses to potentials ranging from −120 mV to −10 mV.

3) Compounds were applied at a holding potential at which 20-50% of the channels was inactivated and sodium channel blockage was monitored during 8 ms test pulses at 2 s intervals.

4) After the compounds equilibrated, the voltage-dependence of steady-state inactivation in the presence of compound was determined according to protocol 2) above. Compounds that block the resting state of the channel decrease the current elicited during test-pulses from all holding potentials, whereas compounds that primarily block the inactivated state shift the mid-point of the steady-state inactivation curve. The maximum current at negative holding potentials (Iₘₐₓ) and the difference in the mid-points of the steady-state inactivation curves (ΔV) in control and in the presence of a compound were used to calculate Kᵣ and Kᵢ using the following equations:

$${K}_{r} = \frac{[ Drug ] * {I}_{Max , Drug}}{{I}_{Max , Control} - {I}_{Max , Drug}}$$

$${K}_{i} = \frac{[ Drug ]}{( 1 + \frac{[ Drug ]}{{K}_{r}} ) * {ⅇ}^{\frac{- Δ V}{k}} - 1}$$

In cases where the compound did not affect the resting state, Kᵢ was calculated using the following equation:

$${K}_{i} = \frac{[ Drug ]}{{ⅇ}^{\frac{- Δ V}{k}} - 1}$$

Rat Formalin Paw Test (In Vivo Assay):

Compounds were assessed for their ability to inhibit the behavioral response evoked by a 50 □L injection of formalin (5%). A metal band was affixed to the left hind paw of male Sprague-Dawley rats (Charles River, 200-250 g) and each rat was conditioned to the band for 60 min within a plastic cylinder (15 cm diameter). Rats were dosed with either vehicle or a test compound either before (local) or after (systemic) formalin challenge. For local administration, compounds were prepared in a 1:4:5 vehicle of ethanol, PEG400 and saline (EPEGS) and injected subcutaneously into the dorsal surface of the left hind paw 5 min prior to formalin. For systemic administration, compounds were prepared in either a EPEGS vehicle or a Tween80 (10%)/sterile water (90%) vehicle and were injected i.v. (via the lateral tail vein 15 min after formalin) or p.o. (60 min before formalin). The number of flinches was counted continuously for 60 min using an automated nociception analyzer (UCSD Anesthesiology Research, San Diego, Calif.). Statistical significance was determined by comparing the total flinches detected in the early (0-10 min) and late (11-60 min) phase with an unpaired t-test.

In Vivo Assay Using Rat CFA Model:

Unilateral inflammation was induced with a 0.2 ml injection of complete Freund's adjuvant (CFA: Mycobacterium tuberculosis, Sigma; suspended in an oil/saline (1:1) emulsion; 0.5 mg Mycobacterium/mL) in the plantar surface of the left hindpaw. This dose of CFA produced significant hind paw swelling but the animals exhibited normal grooming behavior and weight gain over the course of the experiment. Mechanical hyperalgesia was assessed 3 days after tissue injury using a Randall-Selitto test. Repeated Measures ANOVA, followed by Dunnett's Post Hoc test.

SNL: Mechanical Allodynia (In Vivo Assay):

Tactile allodynia was assessed with calibrated von Frey filaments using an up-down paradigm before and two weeks following nerve injury. Animals were placed in plastic cages with a wire mesh floor and allowed to acclimate for 15 min before each test session. To determine the 50% response threshold, the von Frey filaments (over a range of intensities from 0.4 to 28.8 g) were applied to the mid-plantar surface for 8 s, or until a withdrawal response occurred. Following a positive response, an incrementally weaker stimulus was tested. If there was no response to a stimulus, then an incrementally stronger stimulus was presented. After the initial threshold crossing, this procedure was repeated for four stimulus presentations per animal per test session. Mechanical sensitivity was assessed 1 and 2 hr post oral administration of the test compound.

The compounds described in this invention displayed sodium channel blocking activity of from about <0.1 μM to about <50 μM in the in vitro assays described above. It is advantageous that the compounds display sodium channel blocking activity of <5 μM in the in vitro assays. It is more advantageous that the compounds display sodium channel blocking activity of <1 μM in the in vitro assays. It is even more advantageous that the compounds display sodium channel blocking activity of <0.5 μM in the in vitro assays. It is still more advantageous that the compounds display sodium channel blocking activity of <0.1 μM in the in vitro assays.

The present compounds can be prepared according to the general schemes provided below as well as the procedures provided in the Examples. The following schemes and Examples further describe, but do not limit, the scope of the invention.

### Methods of Synthesis

Compounds of the present invention can be prepared according to the Schemes provided below as well as the procedures provided in the Examples. The substituents are the same as in the above Formula except where defined otherwise or otherwise apparent to one skilled in the art.

The novel compounds of the present invention can be readily synthesized using techniques known to those skilled in the art, such as those described, for example, in Advanced Organic Chemistry, March, 4^{th} Ed., John Wiley and Sons, New York, N.Y., 1992; Advanced Organic Chemistry, Carey and Sundberg, Vol. A and B, 3^{rd} d Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the compounds of the present invention may be prepared from the chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for the synthesis of compounds of this invention may include one or more steps of protecting group manipulations and of purification, such as, recrystallization, distillation, column chromatography, flash chromatography, thin-layer chromatography (TLC), radial chromatography and high-pressure chromatography (HPLC). The products can be characterized by using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (LC-MS). Methods of protecting group manipulation, purification, structure identification and quantification are well known to one skilled in the art of chemical synthesis.

Compounds of the present invention can be prepared using one or more methods outlined in the following schemes.

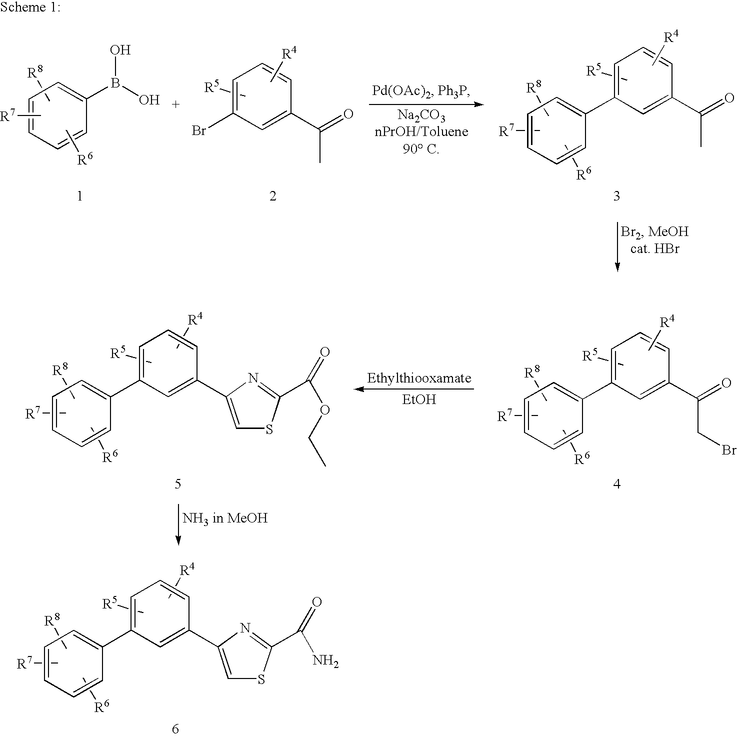

Intermediate 3 can be prepared by reacting aryl ketone 2 with an appropriate phenyl boronic acid 1 under Suzuki Reaction conditions. In a Suzuki reaction, an aryl bromo, iodo, or triflate is reacted with an aryl boronic acid in the presence of a palladium catalyst such as palladium acetate with triphenyl phosphine, and an aqueous sodium carbonate in a solvent such as toluene and a co-solvent such as n-propanol (Suzuki et. al. Chem. Rev., 95, 2457, 1995). A variety of aryl boronic acids are commercially available or can be prepared conveniently from the corresponding aryl bromide or iodide by converting it to an organolithium derivative [Baldwin, J. E. et al. Tetrahedron Lett. 39, 707-710 (1998)], or a Grignard reagent followed by treatment with trialkylborate [Li, J. J. et al, J. Med. Chem, 38: 4570-4578 (1995) and Piettre, S. R. et al. J. Med Chem. 40, 4208-4221 (1997)]. Ketone 3 can be converted into a bromo-ketone 4, which on treatment with ethylthiooxamate can provide the thiazole ester 5. Final treatment with ammonia or a suitable amine can produce the corresponding amide 6 or its analogs.

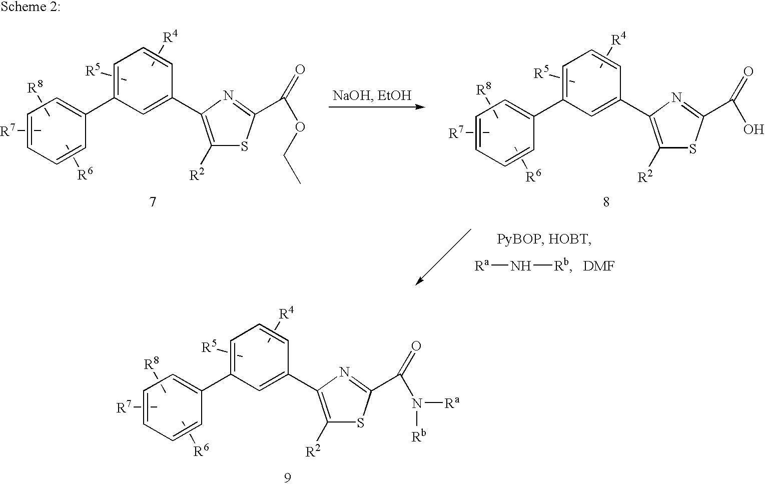

An ester 7 can be hydrolyzed to the corresponding acid 8 which then can be reacted with carbonyldiimidazole (CDI) in DMF, followed by ammonium acetate or an appropriate amine to give the amide 9. The amide 9 can also be prepared from the acid 8 using PyBOP, HOBt combination as the activating agent followed by the addition of an appropriate amine.

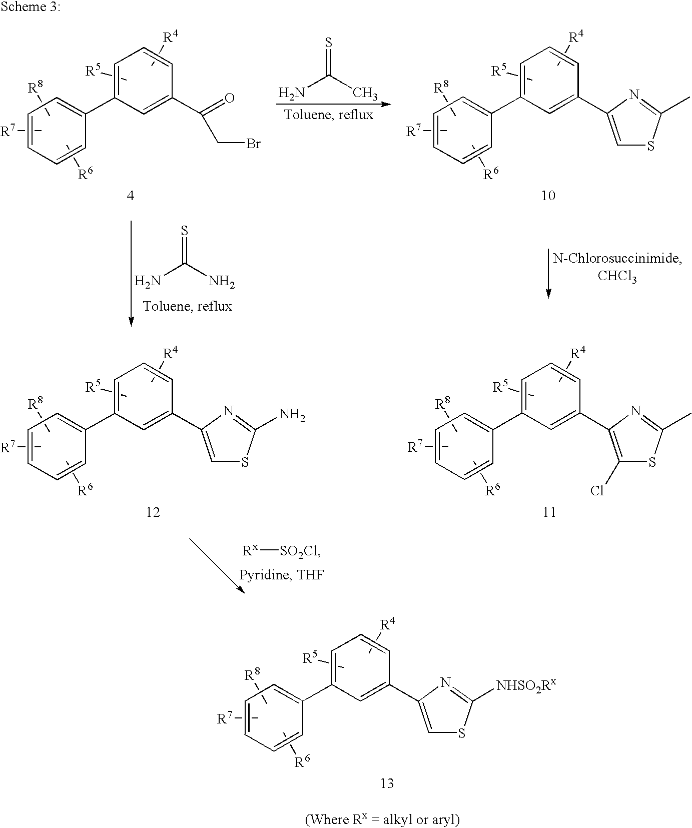

The chloro-thiazole 11 and the amino thiazole 12 can be prepared as outlined in 3. The amino thiazole 12 can be also used in the preparation of sulfonamide 13.

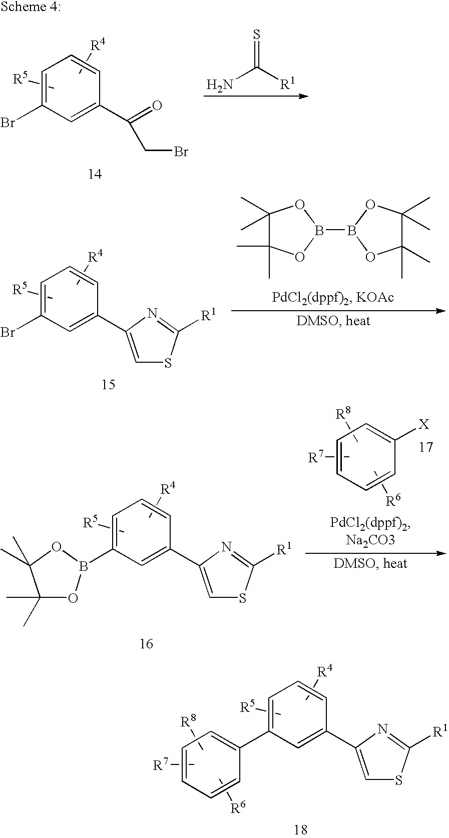

The biaryl compound 18 can be also prepared by forming an aryl boronate 16 from the corresponding bromo compound such as 15, as outlined in 4. Aryl boronates can be used as an alternative to aryl boronic acids in these Pd-catalyzed coupling reactions [Giroux, A. et. al., Tetrahedron Lett., 38, 3841 (1997)]. The boronates can be easily prepared from the aryl bromides, iodides and trifluoromethane sulfonates using the method described by Murata, M. et. al. [J. Org. Chem. 65: 164-168 (2000)]. The chemistry described above can be also accomplished by forming the boronates in situ, followed by their coupling with an appropriate aryl halide 17 under microwave heating to provide 18.

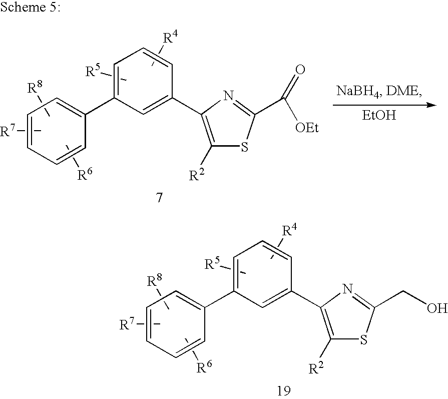

The ester group in 7 can be reduced with an appropriate reducing agent, such as NaBH₄, to provide the corresponding alcohol 19 (5), which can be transformed into a variety of derivatives of 19 using standard chemical transformations known to one skilled in the art.

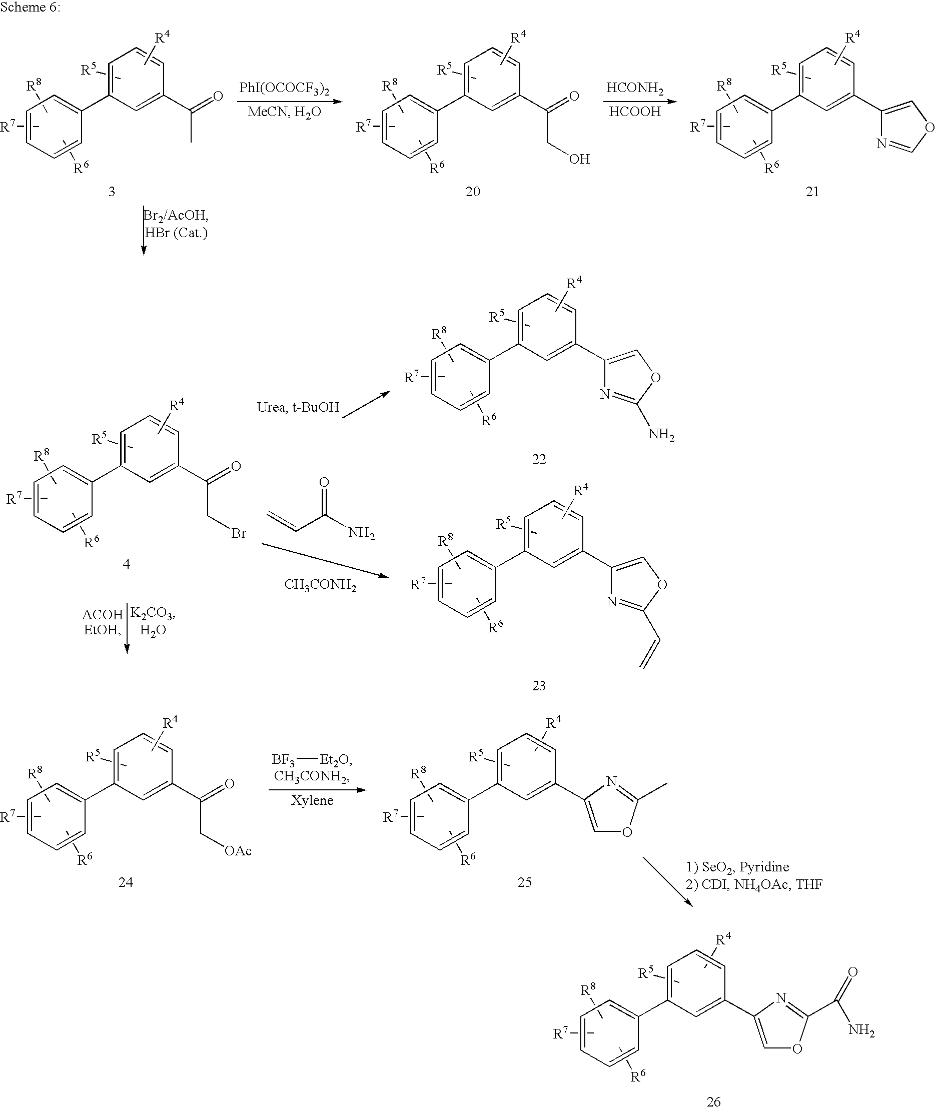

The oxazole compounds of this invention can be prepared as summarized in 6. The α-hydroxyketone 20 obtained from ketone 3 can be reacted with formamide in formic acid to provide oxazole 20. Similarly, oxazoles 22 and 23 can be prepared from α-bromoketone 4. The bromoketone 4 also can be converted into oxazoles 25 and 26 via the acetate derivative 24.

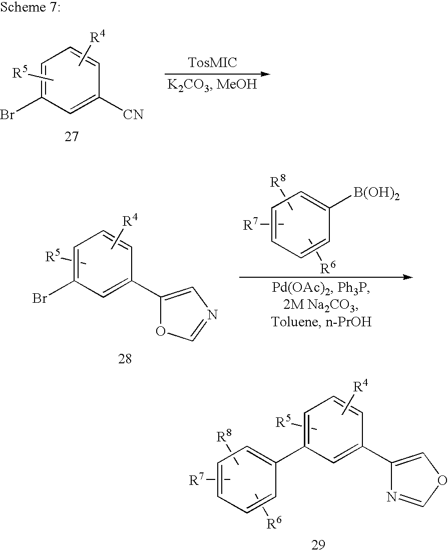

The isomeric oxazole compounds can be synthesized as outlined in 7.

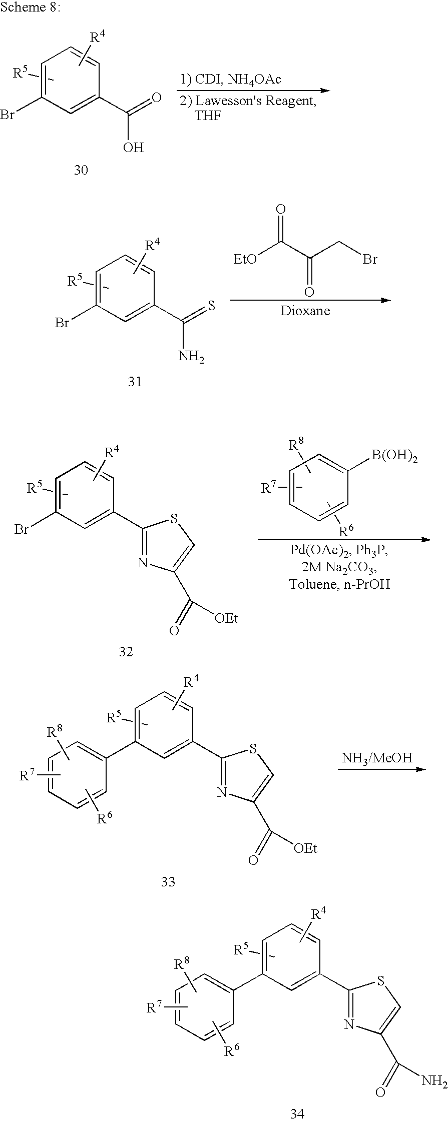

A typical synthesis of thiazole 4-carboxamides 34 is outlined in SCHEME 8. Reaction of an appropriate thioamide 31 with bromo-ethylpyruvate can provide the corresponding thiazole-4-carboxylic acid ester 32, which can be further elaborated as described to produce 34. Furthermore, the ester 33 can be converted into a variety of derivatives using the various methods for functional group transformations know to one skilled in the art.

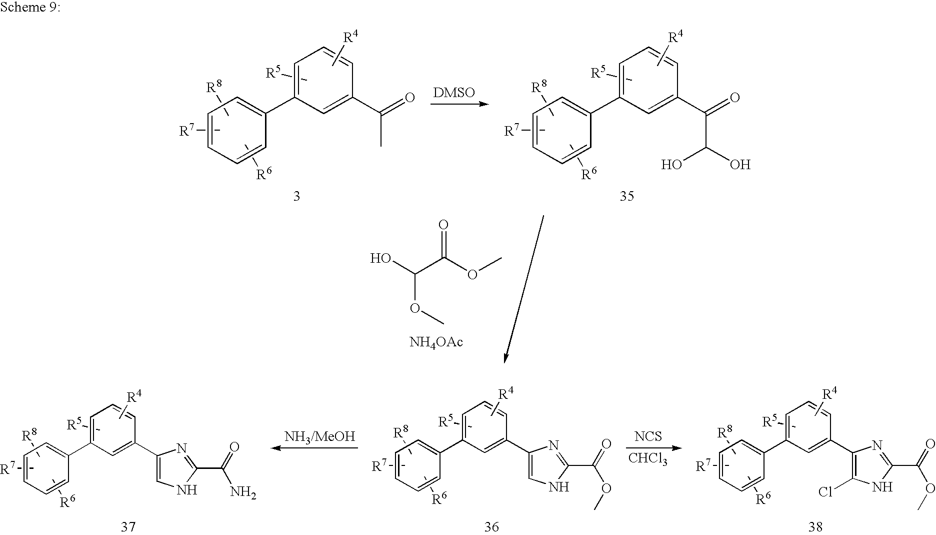

A synthesis of imidazoles 36-38 is outlined in 9.

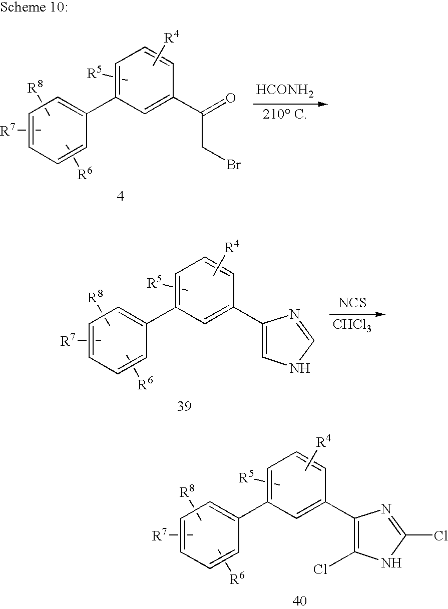

The imidazoles 39 and 40 can be prepared from the α-bromoketone 4, as described in 10.

Appropriate solvents are those in which one or all of the reactants will at least partially be soluble and will not adversely interact with either the reactants or the product. Suitable solvents are aromatic hydrocarbons (e.g, toluene, xylenes), halogenated solvents (e.g, methylene chloride, chloroform, carbontetrachloride, chlorobenzenes), ethers (e.g, diethyl ether, diisopropylether, tert-butyl methyl ether, diglyme, tetrahydrofuran, dioxane, anisole), nitriles (e.g, acetonitrile, propionitrile), ketones (e.g, 2-butanone, dithyl ketone, tert-butyl methyl ketone), alcohols (e.g, methanol, ethanol, n-propanol, iso-propanol, n-butanol, t-butanol), dimethyl formamide (DMF, dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, calcium hydroxide, alkali metal hydrides and alkaline earth metal hydrides such as lithium hydride, sodium hydride, potassium hydride and calcium hydride, alkali metal amides such as lithium amide, sodium amide and potassium amide, alkali metal carbonates and alkaline earth metal carbonates such as lithium carbonate, sodium carbonate, Cesium carbonate, sodium hydrogen carbonate, cesium hydrogen carbonate, alkali metal alkoxides and alkaline earth metal alkoxides such as sodium methoxide, sodium ethoxide, potassium tert-butoxide and magnesium ethoxide, alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, 4-dimethylaminopyridine and also bicyclic amines such as DBU and DABCO.

As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are obviously employed. If desired, tablets may be coated by standard aqueous or nonaqueous techniques. In addition to the common dosage forms set out above, controlled release means and/or delivery devices may also be used in administering the instant compounds and compositions.

It is understood that the functional groups present in compounds described in the above schemes can be further manipulated, when appropriate, using the standard functional group transformation techniques available to those skilled in the art, to provide desired compounds described in this invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. Melting points are uncorrected and ‘d’ indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### EXAMPLE 1

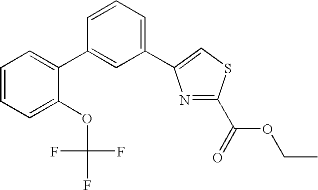

### Ethyl 4-[2′-(trifluoromethoxy)-1,1′-biphenyl-3-yl]-1,3-thiazole-2-carboxylate

### Step 1: Preparation of

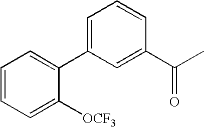

### 1-(2′-trifluoromethoxy-1,1′-biphenyl-3-yl)ethanone

To a solution of 2-bromo(trifluoromethoxy)benzene (4.82 g, 20 mmol) in n-propanol (35 mL) was added 3-acetylbenzeneboronic acid (3.61 g, 22 mmol) under N₂. After 15 min. of stirring at room temperature, Ph₃P (0.46 g, 1.7 mmol) was added followed by 2M sodium carbonate (11 mL)and water (10 mL). To the well stirred solution, palladium acetate (50 mg) was added quickly, and the mixture was refluxed for 4 hours. The reaction was cooled to room temperature and partitioned between EtOAc and water. The organic phase was dried over sodium sulfate and concentrated in vacuo. The crude product obtained was purified by column chromatography on silica gel using 5% EtOAc in hexanes to yield the pure ketone as an oil. Yield: 4.45 g (79%).

¹H-NMR (CDCl₃)(δ, ppm): 8.09 (s, 1H), 8.06 (d, 1H), 7.71 (d, 2H), 7.58 (t, 1H), 7.50-7.40(m, 4H), 2.67 (s, 3H).

MS(ESI): m/e 281 (M+1)⁺.

### Step 2

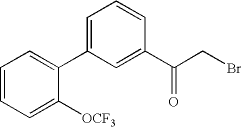

### 2-bromo-1-[2′-(trifluoromethoxy)-1,1′-biphenyl-3-yl]ethanone

To a solution of 1-(2′-trifluoromethoxy-1,1′-biphenyl-3-yl)ethanone (1.0 g, 3.5 mmol) in methanol (7.8 ml), 3 drops of hydrobromic acid was added followed by dropwise addition of solution of bromine (232 ml, 4.52 mmol) in 1 ml of methanol. After the addition, the reaction mixture was stirred at room temperature for 16 hours. The solution was then partitioned between ethyl acetate and water, washed with brine, dried over sodium sulfate, filtered and concentrated to give 2-bromo-1-[2′-(trifluoromethoxy)-1,1′-biphenyl-3-yl]ethanone (958 mg, 75% yield), which was used as such in the subsequent step.

### Step 3: Ethyl 4-[2′-trifluoromethoxy)-1,1′-biphenyl-3-yl]-1,3-thiazole-2-carboxylate

To a solution of the bromide (from Step 2 above) (0.415 g, 1.1 mmol) in ethanol (3.8 ml) was added ethyl thioxamate (0.169 g, 1.27 mmol), and the mixture was refluxed for 16 hours. The reaction was then cooled and partitioned between ethyl acetate and water. The organic phase was washed with saturated sodium bicarbonate, water, then dried, filtered and concentrated. The crude product was purified by silica-gel column chromatography using 10% EtOAc in hexanes to give the titled product (0.395 g, 86%) as a syrup.

¹HNMR (CDCl₃)(δ, ppm): 8.05 (s, 1H), 8.01 (d, 1H), 7.80 (s, 1H), 7.55-7.50 (m, 3H), 7.44-7.40 (m, 3H), 4.54 (q, 2H), 1.49 (t, 3H).

MS (ESI): m/e 394.1 (M+1)⁺.

### EXAMPLE 2

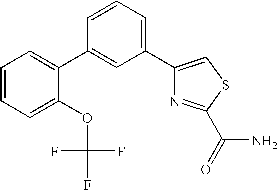

### 4-(2-trifluoromethoxy-1,1′-biphenyl-3yl)-1,3-thiazole-2-carboxamide

Ammonia was bubbled into a solution of ethyl 4-[2′-trifluoromethoxy)-1,1′-biphenyl-3-yl]-1,3-thiazole-2-carboxylate (from EXAMPLE 1) (0.125 g, 0.3 mmol) in methanol (1 ml) at 0° C., and the solution was placed a sealed tube and stirred for 16 hours at room temperature. The reaction was then concentrated in vacuo, and the crude product was purified by silica-gel column chromatography using 40% EtOAc in hexanes to give the product as a solid (0.048 g, 41%).

¹HNMR (CDCl₃)(δ, ppm): 8.03 (s, 1H), 7.93 (d, 1H), 7.80 (s, 1H), 7.56-7.50 (m, 3H), 7.46-7.40 (m, 3H), 5.9 (br s, 2H).

MS (ESI): m/e 365.1 (M+1)⁺.

The following EXAMPLES (summarized in TABLE 1) of this invention were prepared according to the methods described in EXAMPLES 1 and 2.

TABLE 1

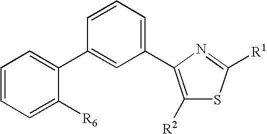

| EXAMPLE | | | | MS (m/e, |
|---|---|---|---|---|
| # | R⁶ | R² | R¹ | M + 1)⁺ |
| 3 | Cl | H | H | 272.0 |
| 4 | Cl | H | COOEt | 343.9 |
| 5 | Cl | H | CONH₂ | 315.0 |
| 6 | Cl | H | CONH-tBu | 371.0 |
| 7 | Cl | H | 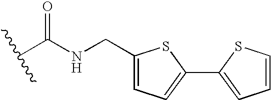 | 493.0 |
| 8 | Cl | H | NH₂ | 287.0 |
| 9 | CF₃ | H | COOEt | 378.0 |
| 10 | CF₃ | H | CONH₂ | 349.0 |
| 11 | CF₃ | H | H | 305.9 |
| 12 | CF₃ | H | NH₂ | 321.0 |
| 13 | OCF₃ | H | CH₃ | 336.0 |
| 14 | OCF₃ | H | H | 322.0 |
| 15 | OCF₃ | H | NH₂ | 337.1 |
| 16 | OCF₃ | H | CONMe₂ | 393.0 |
| 17 | OCF₃ | Cl | CH₃ | 370.1 |
| 18 | OCF₃ | H | NHSO₂CH₃ | 414.9 |
| 19 | OCF₃ | H | CH₂OH | 352.0 |
| 20 | O—Ph | H | CONH₂ | 373.0 |
| 21 | CF₃ | H | NHCONH-iPr | 406.2 |
| 22 | OCF₃ | H | NHCONH-iPr | 422.1 |
| 23 | OCF₃ | H | NHCOCH₃ | 378.9 |
| 24 | CF₃ | H | NHCOCH₃ | 363.0 |
| 25 | OCF₃ | H | CH₂COOEt | 408.0 |
| 26 | OCF₃ | H | CH₂CN | 361.0 |
| 27 | OCF₃ | H | CH₂CONH₂ | 379.0 |
| 28 | CF₃ | H | CH₂CONH₂ | 362.9 |
| 29 | OCF₃ | H | NHCONMe₂ | 408.0 |
| 30 | OCF₃ | H | 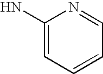 | 441.9 |
| 31 | OCF₃ | H | 2-Pyrimidyl | 400.1 |
| 32 | OCF₃ | H | 2-Pyridyl | 399.0 |
| 33 | OCF₃ | H | 2-Oxazolyl | 389.0 |
| 34 | OCF₃ | H | 2-Imidazolyl | 387.2 |
| 35 | OCF₃ | H | 2-Pyrazolyl | 387.0 |
| 36 | OCF₃ | H | 2-(1-Methyl)- | 400.9 |
| | | | imidazolyl | |
| 37 | OCF₃ | H | 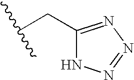 | 404.0 |
| 38 | OCF₃ | H | 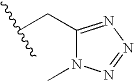 | 418.0 |
| 39 | OCF₃ | H | 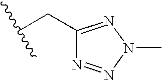 | 418.0 |

Further EXAMPLES of this invention are shown in TABLE 2.

TABLE 2

| EXAM- | | MS |
|---|---|---|
| PLE | | (m/e, |
| # | STRUCTURE | M + 1) |
| 40 | 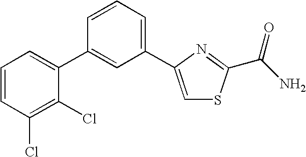 | 348.8 |
| 41 | 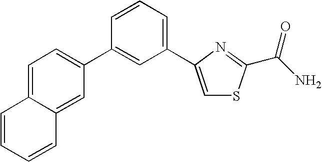 | 331.0 |

### EXAMPLE 42

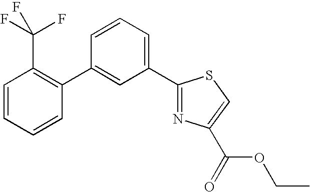

### Ethyl 2-[2′-(trifluoromethyl)-1,1′-biphenyl-3-yl]-1,3-thiazole-4-carboxylate

To a solution of 3-bromobenzamide (0.81 g, 4 mmol) in tetrahydrofuran (5 ml) was added Lawesson's reagent (1.79 g, 4.4 ml) and stirred at room temperature for 16 hours. The reaction mixture was concentrated in vacuo, and the crude product was purified by column chromatography using 10% EtOAc in hexanes to give 3-bromothiobenzamide (0.625 g, 71% yield) as a yellow solid.

To a solution of the thioamide (0.50 g, 2.31 mmol) in dioxane (4 ml) was added ethyl bromopyruvate (0.587 g, 3.01 mmol). The mixture refluxed for 16 hours, then cooled to room temperature and partitioned between ethyl acetate and water. The organic phase was washed with saturated sodium bicarbonate and water, then dried over sodium sulfate, filtered and concentrated. Purification of the crude by column chromatography using 10% EtOAc in hexanes yielded (0.45 g, 62% ) of the ethyl 2-(3-bromophenyl)-1,3-thiazole-4-carboxylate as a syrup.

To a solution of the above ester (0.149 g, 0.4 mmol) in toluene (1.5 ml) were added 2-trifluromethylphenylboronic acid (0.135 g, 0.71 mmol), tetrakis(triphenylphosphine) palladium (0.01 g) and 2 M potassium carbonate (0.48 ml, 0.95 mmol). The reaction was heated at 85° C. for 16 hours in a sealed tube, then cooled to room temperature and partitioned between ethyl acetate and saturated sodium bicarbonate. The organic phase was washed with water, brine, dried over sodium sulfate, filtered and concentrated. Purification of the crude by column chromatography using 10% EtOAc in hexanes gave ethyl 2-[2′-(trifluoromethyl)-1,1′-biphenyl-3-yl]-1,3-thiazole-4-carboxylate (0.14 g, 89%) as a syrup.

¹HNMR (CDCl₃)(δ, ppm): 8.19 (s, 1H), 8.03 (d, 1H), 8.01 (s, 1H), 7.79 (d, 1H0, 7.61 (t, 1H), 7.60-7.52 (m, 2H), 7.45 (d, 1H), 7.41 (d, 1H), 4.47 (q, 2H), 1.45 (t, 1H).

MS (ESI): 378.0 (M+1)⁺.

### EXAMPLE 43

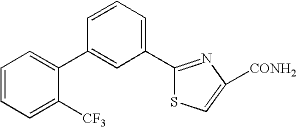

### 2-[2′-(trifluoromethyl)-1,1′-biphenyl-3-yl]-1,3-thiazole-4-carboxamide

The titled compound was prepared by reacting ethyl 2-[2′-(trifluoromethyl)-1,1′-biphenyl-3-yl]-1,3-thiazole-4-carboxylate (0.068 g, 0.2 mmol) with ammonia in methanol (1 ml), as described in EXAMPLE 2. The crude product was purified by flash chromatography on silica-gel using 30% EtOAc in hexanes to give the pure product (0.048 g, 68%) as a solid.

¹HNMR (CDCl₃)(δ, ppm): 8.1 (s, 1H), 8.05 (d, 1H), 7.9 (s, 1H), 7.81 (d, 1H), 7.64 (t, 1H), 7.55-7.52 (m, 2H), 7.47 (d, 1H), 7.41 (d, 1H), 7.26 (br s, 1H), 5.8 (br s, 1H).

MS (ESI): 349 (M+1)⁺.

The following EXAMPLES of this invention were prepared according to procedures described in EXAMPLES 42 and 43, and are summarized below in TABLE 3.

TABLE 3

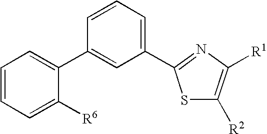

| EXAMPLE | | | | MS (m/e, |
|---|---|---|---|---|
| # | R₆ | R₂ | R₁ | M + 1) |
| 44 | CF₃ | H | H | 306.1 |
| 45 | CF₃ | H | COOEt | 378.0 |
| 46 | CF₃ | H | CONH₂ | 348.9 |
| 47 | CF₃ | H | CONHCH₃ | 379.0 |
| 48 | CF₃ | COOEt | CH₃ | 392.0 |
| 49 | CF₃ | CONH₂ | CH₃ | 362.1 |
| 50 | OCF₃ | H | H | 322.1 |
| 51 | OCF₃ | H | COOCH₃ | 379.9 |
| 52 | OCF₃ | H | CONH₂ | 365.0 |
| 53 | OCF₃ | H | COOH | 322.0 (M − |
| | | | | 44 + 1) |
| 54 | OCF₃ | H | CH₂OH | 352.0 |
| 55 | OCF₃ | H | CONH(CH₂)₃OH | 423.1 |
| 56 | O—Ph | H | CONH₂ | 373.1 |

### EXAMPLE 57

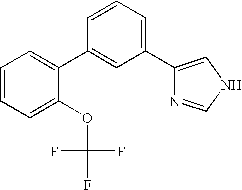

### 5-[2′-(trifluoromethoxy)-1,1′-biphenyl-3-yl]-1H-imidazole

A solution of bromine (0.232 ml, 4.52 mmol) in methanol (1 ml) was added to a solution of 1-(2′-trifluoromethoxy-1,1′-biphenyl-3-yl)ethanone (1.0 g, 3.5 mmol) (from Step 1, EXAMPLE 1) in methanol (7.8 ml) containing 3 drops of hydrobromic acid, and the reaction was stirred at room temperature for 16 hours. The reaction was then partitioned between ethyl acetate and water, and the organic phase was washed with water and brine, then dried over sodium sulfate, filtered and concentrated to give 2-bromo-1-[2′-(trifluoromethoxy)-1,1′-biphenyl-3-yl]ethanone (0.958 g, 75% yield).

A solution 1-[2′-trifluoromethoxy-1,1′-biphenyl-3-yl)ethanone (155 mg, 0.5 mmol) in formamide was heated at 230° C. for 700 secs using a Smith Creator™ microwave reactor (commercially available from Personal Chemistry, Inc.). The solution was cooled and partitioned between ethyl acetate & water. The organic phase was washed with brine, dried over sodium sulfate, filtered and concentrated. The residue was purified by column chromatography using 10% EtOAc in hexanes to give the product (0.088 g) in 53% yield.

¹HNMR (CDCl₃)(δ, ppm): 8.5 (s, 1H), 7.75 (s, 1H), 7.67(d, 1H), 7.67-7.50 (m, 2H), 7.41-7.34 (m, 1H), 7.16 (d, 1H).

MS(ESI): 305.1 (M+1)⁺.

### EXAMPLE 58

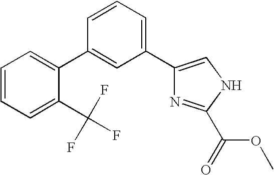

### Methyl 5-[2′-(trifluoromethyl)-1,1′-biphenyl-3-yl]-1H-imidazole-2-carboxylate

### Step 1

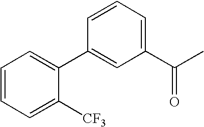

### 1-(2′-trifluoromethyl-1,1′-biphenyl-3-yl)ethanone

The titled compound was prepared in 78% yield according to the procedure described in Step 1 of EXAMPLE 1 using 2-trifluoromethyl-phenylboronic acid.

¹HNMR (CDCl₃)(δ, ppm): 8.02 (d, 1H), 7.95 (s, 1H), 7.81 (d, 1H), 7.61 (t, 1H), 7.57-7.53 (m, 3H), 7.36 (d, 1H), 2.66 (s, 3H).

MS (ESI): 265.1 (M+1)⁺.

### Step 2: Methyl 5-[2′-(trifluoromethyl)-1,1′-biphenyl-3-yl]-1H-imidazole-2-carboxylate

To a solution of the ketone from Step 1 (0.473 g, 1.8 mmol) in DMSO (2.4 ml) was added 48% HBr (0.14 ml) at 60° C. and stirred at that temperature for 16 hours. After cooling, the reaction was partitioned between ethyl acetate and water. The organic phase was washed with brine, dried over sodium sulfate, filtered and concentrated to give 2,2-dihydroxy-1-[2′-(trifluoromethyl)-1,1′-biphenyl-3-yl]ethanone (0.530 g) which was used as such for the next step.

To a solution of ammonium acetate (0.411 g, 5.3 mmol) in water (5 ml) and acetonitrile (5 ml) at 0° C. was added methyl glyoxate (0.644 g, 5.3 mmol) followed by 2,2-dihydroxy-1-[2′-(trifluoromethyl)-1,1′-biphenyl-3-yl]ethanone (0.53 g, 1.78 mmol) in acetonitrile (2.8 ml) over a period of 20 min at 0° C. The mixture was stirred at 0-5° C. for 30 min and at room temperature for 1 hour, then partitioned between ethyl acetate and water. The organic layer was washed with brine and dried over sodium sulfate, filtered and concentrated. The residue obtained was purified by silica-gel column chromatography using (2.5:2.5:0.1 hexanes/ethyl acetate/2N ammonia in methanol) to give the product (0.54 g, 87%) as a syrup.

¹HNMR (CDCl₃)(δ, ppm): 7.78 (d, 1H), 7.73 (d, 1H), 7.68(s, 1H), 7.62-7.59 (m, 2H), 7.57-7.47 (m, 2H), 7.39 (d, 1H), 7.36 (d, 1H), 5.3 (br s, 2H), 3.96 (s, 3H).

MS (ESI): m/e 347.1 (M+1)⁺.

### EXAMPLE 59

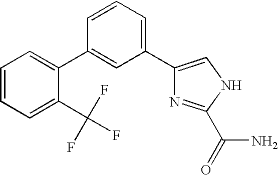

### 5-[2′-(trifluoromethyl)-1,1′-biphenyl-3-yl]-1H-imidazole-2-carboxamide

Methyl 5-[2′-(trifluoromethyl)-1,1′-biphenyl-3-yl]-1H-imidazole-2-carboxylate from EXAMPLE 58 (0.213 g, 0.6 mmol) was mixed in a sealed tube with a saturated solution of ammonia in methanol (1 ml) and stirred at room temperature for 3 days. The crude product obtained, after removal of excess reagent and the solvent in vacuo, was purified by column chromatography (2.5:2.5:0.1 hexanes/ethyl acetate/ammonia in methanol) to give the titled product (0.12 g, 59%) as a solid, which was converted into the hydrochloride salt.

¹HNMR (CD₃OD)(δ, ppm): 7.84-7.76 (m, 3H), 7.66-7.43 (m, 4H), 7.31(s, 1H).

MS (ESI): m/e 332.0 (M+1)⁺.

The following EXAMPLES (TABLE 4) were prepared according to methods described in EXAMPLES 60 and 61.

TABLE 4

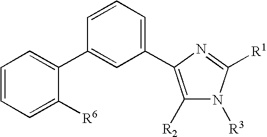

| EXAMPLE | | | | | MS (m/e, |
|---|---|---|---|---|---|
| # | R⁶ | R³ | R² | R¹ | M + 1) |
| 60 | Cl | H | H | H | 255.0 |
| 61 | Cl | H | H | COOCH₃ | 312.9 |
| 62 | Cl | H | H | CONH₂ | 298.0 |
| 63 | CF₃ | H | H | H | 289.0 |
| 64 | CF₃ | H | H | COOH | 333.0 |
| 65 | CF₃ | CH₃ | H | CONH₂ | 345.9 |
| 66 | CF₃ | H | Cl | COOCH₃ | 380.9 |
| 67 | CF₃ | H | Cl | CONH₂ | 366.1 |
| 68 | CF₃ | H | Cl | Cl | 357.0 |
| 69 | OCF₃ | H | H | H | 305.2 |
| 70 | OCF₃ | H | H | COOCH₃ | 363.0 |
| 71 | OCF₃ | H | H | CONH₂ | 348.1 |
| 72 | OCF₃ | H | H | 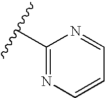 | 383.0 |

### EXAMPLE 73

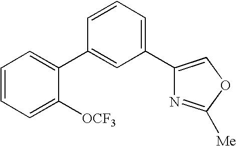

### 2-Methyl-4-[2′-(trifluoromethoxy)-1,1′-biphenyl-3-yl]-1,3-oxazole

### Step 1: 2-oxo-2-[2′-(trifluoromethoxy)-1,1′-biphenyl]-3-yl]ethyl acetate

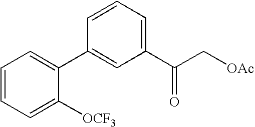

To a solution of the methyl ketone from Step 1 of EXAMPLE 1 (0.730 g, 2.6 mmol) and hydrogen bromide (0.1 ml) in methanol (10 mL) was added bromine (0.17 ml, 3.4 mmol) dropwise. The resulting dark red solution was stirred at room temperature for 20 hours. The solvent was removed under reduced pressure. The residue was dissolved in ethyl acetate and washed with saturated sodium bicarbonate, brine, and dried over anhydrous sodium sulfate. The pure α-bromo ketone was isolated as a white solid, (0.93 g, 99%) after column chromatography on silica gel.

To a solution of acetic acid (18 mg, 0.31 mmol) in a mixture of methanol and water (5:1, 3 mL) was added the potassium carbonate powder (0.043 g, 0.31 mmol). After about 10 minutes, the α-bromo ketone (from above) (0.1 g, 0.28 mmol) was added and the mixture was refluxed for 3 hours. After cooling to room temperature the solvent was removed under reduce pressure. The residue was dissolved in ethyl acetate, and washed with water, brine, and dried over anhydrous sodium sulfate. The pure titled product was isolated as a white solid (0.050 g, 53%) after column chromatography on silica gel.

¹H-NMR (CDCl₃) (δ, ppm): 7.98 (s, 1H), 7.92 (d, J=6.5 Hz, 1H), 7.70 (d, J=6.5 Hz, 1H), 7.55 (t, J=7.5 Hz, 1H), 7.39 (m, 4H), 5.35 (s, 2H), 2.22 (s, 3H).

MS (ESI): m/e 339.0 (M+1)⁺

### Step 2: 2-Methyl-4-[2′-(trifluoromethoxy)-1,1′-biphenyl-3-yl]-1,3-oxazole

To a solution of the acetate (from Step 1) (0.05 g, 0.15 mmol) in xylene (3 mL) was added acetamide (35 mg, 0.59 mmol) and boron trifluoride etherate (0.018 mL, 0.15 mol). The resulting colorless solution was refluxed for 18 hours. After cooling to room temperature, and removing the solvent, the residue was partitioned between ethyl acetate and saturated sodium bicarbonate. The aqueous phase was extracted with ethyl acetate, and the combined organic layer was washed with brine, then dried over anhydrous sodium sulfate. The pure titled product was isolated as a white solid (15 mg, 32%) after column chromatography on silica gel.

¹H-NMR (CDCl₃) (δ, ppm): 7.82 (s, 1H), 7.78 (s, 1H), 7.70 (d, J=4.5 Hz, 1H), 7.47-7.34 (m, 6H), 2.50 (s, 3H).

MS (ESI): m/e 320.1 (M+1)⁺

### EXAMPLE 74

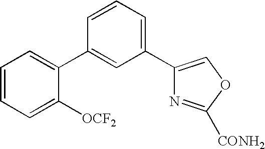

### 4-[2′-(trifluoromethoxy)-1,1′-biphenyl-3-yl]-1,3-oxazole-2-carboxamide

To a solution of the methyl oxazole (from EXAMPLE 73) (0.16 g, 0.5 mmol) in pyridine (3 mL) was added selenium dioxide (0.56 g, 5 mmol) and the resulting colorless solution was refluxed for 18 hours. The reaction turned yellow with black precipitate after a few hours of refluxing. After cooling, the solvent was removed in vacuo, and the residue was partitioned between ethyl acetate and 1N HCl aqueous solution. The aqueous phase was extracted with ethyl acetate. The combined organic layer was washed with brine and dried over anhydrous sodium sulfate. The crude carboxylic acid obtained was immediately dissolved in dry THF (5 mL), and 1.1′-carbonyldiimidazole (0.065 g, 0.4 mmol) was added. After 1 hour of stirring at room temperature, ammonium acetate (0.31 g, 4 mmol) was added, and the mixture was stirred for 2 days. After removing the solvent, the residue was dissolved in ethyl acetate, washed with saturated ammonium chloride solution, brine, and dried over anhydrous sodium sulfate. The pure titled compound was isolated as a yellow solid (0.083 g, 47%), after column chromatography on silica gel.

¹H-NMR (CDCl₃) (δ, ppm): 7.89 (s, 1H), 7.81(d, J=7.5 Hz, 1H), 7.62 (d, J=7.5 Hz, 1H), 7.48 (t, J=8.0 Hz, 1H), 7.43-7.35 (m, 4H), 6.26 (bs, 2H).

MS (ESI): m/e 349.1 (M+1)⁺

The following EXAMPLES (TABLE 5) were prepared according to methods described in EXAMPLES 73 and 74.

TABLE 5

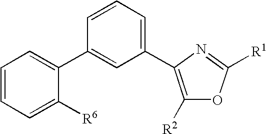

| EXAMPLE | | | | MS (m/e, |
|---|---|---|---|---|
| # | R⁶ | R² | R¹ | M + 1) |
| 75 | Cl | H | CH₃ | 270.0 |
| 76 | Cl | H | NH₂ | 271.0 |
| 77 | CF₃ | H | CH₃ | 304.1 |
| 78 | OCF₃ | H | NH₂ | 321.0 |
| 79 | OCF₃ | H | CH═CH₂ | 332.0 |
| 80 | OCF₃ | H | COOCH₃ | 363.9 |
| 81 | OCF₃ | H | CONH₂ | 349.0 |
| 82 | OCF₃ | H | CH₃ | 320.0 |
| 83 | CF₃ | H | NH₂ | 305.1 |
| 84 | CF₃ | H | H | 289.9 |
| 85 | OCF₃ | H | H | 306.0 |
| 86 | Cl | H | COOH | 300.0 |
| 87 | CF₃ | H | CONH₂ | 333.1 |
| 89 | OCF₃ | H | COOCH₂CH₃ | 378.0 |

### EXAMPLE 90

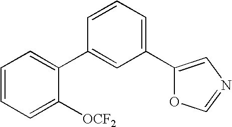

### 5-[2′-(trifluoromethoxy)-1,1′-biphenyl-3-yl]-1,3-oxazole

### Step 1: 5-(3-bromophenyl)-1,3-oxazole

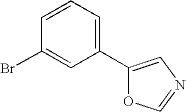

To a solution of the 3-bromo benzaldehyde (0.2 g, 1.1 mmol) and TosMic (0.205 g, 1.1 mmol) in dry methanol was added potassium carbonate powder (0.145 g, 1.1 mmol). The resulting mixture was refluxed for 2 hours. After cooling to room temperature, the solvent was removed under reduced pressure. The residue was partitioned between ethyl acetate and water. The aqueous was extracted with ethyl acetate, and the combined organic layer was washed with brine, then dried over anhydrous sodium sulfate and concentrated to give the titled product as a yellow solid (0.225 g, 93%).

¹H-NMR (CDCl₃) (δ, ppm): 7.90 (s, 1H), 7.78 (s, 1H), 7.55 (d, J=7.5 Hz, 1H), 7.43 (d, J=7.5 Hz, 1H), 7.35 (s, 1H), 7.27 (m, 1H).

MS (ESI): m/e 223.9 (M+1)⁺.

### Step 2: 5-[2′-(trifluoromethoxy)-1,1′-biphenyl-3-yl]-1,3-oxazole

To a solution of the product from Step 1 (0.225 g, 1 mmol) and 2-trifluoromeoxyphenylboronic acid (0.267 g, 1.4 mmol) in n-propanol (20 mL), were added palladium acetate (22.5 mg, 0.1 mmol), triphenyl phosphine (79 mg, 0.3 mmol), and aqueous sodium carbonate (2.0M, 0.6 mL, 1.2 mmol). The mixture was stirred at 90° C. for 16 hours, and then cooled to room temperature, filtered through a pad of Celite, and washed with ethyl acetate (3 times). The filtrate was washed with saturated sodium bicarbonate aqueous solution, brine, then dried over anhydrous sodium sulfate and concentrated in vacuo. The titled product was obtained as a white solid (0.2 g, 69%), after column chromatography.

¹H-NMR (CDCl₃) (δ, ppm): 7.91 (s, 1H), 7.74 (s, 1H), 7.66 (d, J=7.5 Hz, 1H), 7.50-7.35 (m, 7H).

MS (ESI): m/e 306.0 (M+1)⁺

Other variations or modifications, which will be obvious to those skilled in the art, are within the scope and teachings of this invention. This invention is not to be limited except as set forth in the following claims.

What is claimed is:

1. A compound represented by Formula (I): 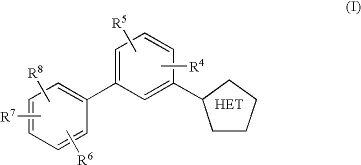
or a pharmaceutically acceptable salt thereof, wherein
HET is one of the following heterocycles: 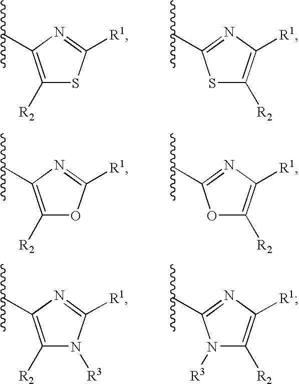
R¹ is
(a) H;
(b) C₁-C₆-alkyl, C₂-C₄-alkenyl, C₂-C₄-alkynyl-[C₃-C₆-cycloalkyl, or C₁-C₄-alkyl-[C₃-C₆-cycloalkyl], any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —O—C₁-C₆-alkyl, —O—C₃-C₆-cycloalkyl, S—C₁-C₆-alkyl or —S—C₃-C₆-cycloalkyl, any of which is optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)allyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(d) —C₀-C₄-alkyl-C₁-C₄-perfluoroalkyl, or —O—C₀-C₄-alkyl-C₁-C₄-perfluoroalkyl;
(e) —OH;
(f) —O-aryl, or —O—C₁-C₄-alkylaryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(g) —OCON(R^{a})(R^{b}), or —OSO₂N(R^{a})(R^{b});
(h) —SH, or —SCON(R^{a})(R^{b});
(i) NO₂;
(j) NR^{a}R^{b}, —N(COR^{a})R^{b}, —N(SO₂R^{a})R^{b}, —N(R^{a})SO₂N(R^{a})₂, —N(OR^{a})CONR^{a}R^{b}, —N(R^{a})SO₂R^{a} or —N(R^{a})CON(R^{a})₂;
(k) —CH(OR^{a})R^{a}, —C(OR^{b})CF₃, —CH(NHR^{b})R^{a}, —C(═O)R^{a}, C(═O)CF₃, —SOCH₃, —SO₂CH₃, COOR^{a}, CN, CONR^{a}R^{b}, —COCONR^{a}R^{b}, —SO₂NR^{a}R^{b}, —CH₂O—SO₂NR^{a}R^{b}, SO₂N(R^{a})OR^{a}, —C(═NH)NH₂, —CR^{a}═N—OR^{a}, CH═CHCONR^{a}R^{b};
(l) —CONR^{a}(CH₂)₀₋₂C(R^{a})(R^{b})(CH₂)₀₋₂CONR^{a}R^{b};
(m) tetrazolyl, tetrazolinonyl, triazolyl, triazolinonyl, imidazolyl, imidozolonyl, oxazolyl, oxadiazolyl, isooxazolyl, thiazolyl, furyl, thienyl, pyrazolyl, pyrazolonyl, pyrrolyl, pyridyl, pyrimidinyl, pyrazinyl, or phenyl, any of which is optionally substituted with 1-3 substituents selected from i) F, Cl, Br, I, ii) —CN, iii) —NO₂, iv) —C(═O)R^{a}, v) C₁-C₆-alkyl, vi) —O—R^{a}, vii) —NR^{a}R^{b}, viii) —C₀-C₄-alkyl —CO—OR^{a}, ix) —(C₀-C₄-alkyl)-NH—CO—OR^{a}, x) —(C₀-C₄-alkyl)-CO—NR^{a}R^{b}, xi) —S(O)₀₋₂R^{a}, xii) —SO₂NR^{a}R^{b}, xiii) —NHSO₂R^{a}, xiv) —C₁-C₄-perfluoroalkyl, and xv) —O—C₁-C₄-perfluoroalkyl;
(n) —C(R^{a})═C(R^{b})—COOR^{a}, or —C(R^{a})═C(R^{b})—CONR^{a}R^{b};
(o) 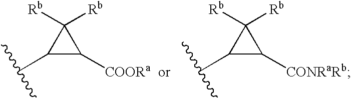
(p) piperidin-1-yl, morpholin-4-yl, pyrrolidin-1-yl, piperazin-1-yl or 4-substituted piperazin-1-yl, any of which is optionally substituted with 1-3 substituents selected from i) —CN, ii) —C(═O)(R^{a}), iii) C₁-C₆-alkyl, iv) —OR^{a}, v) —NR^{a}R^{b}, vi) —C₀-C₄-alkyl-CO—OR^{a}, vii) —(C₀-C₄-alkyl)-NH—CO—OR^{a}, viii) —(C₀-C₄-alkyl)-CON(R^{a})(R^{b}), ix) —SR^{a}, x) —S(O)₀₋₂R^{a}, xi) —SO₂N(R^{a})(R^{b}), xii) —NR^{a}SO₂R^{a} xiii) —C₁-C₄-perfluoroalkyl and xiv) —O—C₁-C₄-perfluoroalkyl;
R^{a} is
(a) H;
(b) C₁-C₄-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), —OCON(C₁-C₄alkyl)(C₁-C₄alkyl), —OCONHC₁-C₄alkyl-aryl), —OCON(C₁-C₄alkyl)(C₁-C₄alkyl-aryl), NH₂, NH(C₁-C₄alkyl), N(C₁-C₄alkyl)(C₁-C₄alkyl), NH(C₁-C₄alkyl-aryl), N(C₁-C₄alkyl)(C₁-C₄alkyl-aryl), NHCONH₂, NHCONH(C₁-C₄alkyl), NHCONH(C₁-C₄alkyl-aryl), —NHCON(C₁-C₄alkyl)(C₁-C₄alkyl), NHCON(C₁-C₄alkyl)(C₁-C₄alkyl-aryl), N(C₁-C₄alkyl)CON(C₁-C₄alkyl)(C₁-C₄alkyl), N(C₁-C₄alkyl)CON(C₁-C₄alkyl)(C₁-C₄alkyl-aryl), COO—(C₁-C₄-alkyl), COOH, CN, CONH₂, CONH(C₁-C₄alkyl), CON(C₁-C₄alkyl)(C₁-C₄alkyl), SO₂NH₂, SO₂NH(C₁-C₄alkyl), SON(C₁-C₄alkyl-aryl), SO₂N(C₁-C₄alkyl)(C₁-C₄alkyl), NHSO₂NH₂, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) C₀-C₄-alkyl-(C₁-C₄)-perfluoroalkyl; or
(d) C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(C₁-C₄-alkyl), v) —O(C₁-C₄-alkyl), vi) —N(C₁-C₄-alkyl)(C₁-C₄-alkyl), vii) —C₁₋₁₀alkyl, and viii) —C₁₋₁₀alkyl, wherein one or more of the alkyl carbons can be replaced by a —O—, —S(O)₁₋₂—, —O—C(O)—, —C(O)—O—, —C(O)—, —CH(OH)—, —C≡C—, or —C≡C—;
R^{b} is
(a) H; or
(b) C₁-C₆-alkyl, optionally substituted with one or more of the following substituents: F, CF₃, OH, O—(C₁-C₄)alkyl, S(O)₀₋₂—(C₁-C₄)alkyl, —OCONH₂, —OCONH(C₁-C₄alkyl), NH₂, NH(C₁-C₄alkyl), N(C₁-C₄alkyl)(C₁-C₄alkyl), NHCONH₂, NHCONH(C₁-C₄alkyl), —NHCON(C₁-C₄alkyl)(C₁-C₄alkyl), COO—(C₁-C₄-alkyl), COOH, CN, and CONH₂;
R² is:
(a) H;
(b) —C₁-C₄-alkyl, —C₃-C₆-cycloalkyl or —C₁-C₄-alkyl-(C₃-C₆)-cycloalkyl, optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a})CONR^{a}R^{b}, COO—(C₁-C₄)alkyl, COOH, CN, CONR^{a}R^{b}, SO₂NR^{a}R^{b}, N(R^{a})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl and piperazinyl;
(c) —C₀-C₄-alkyl-C₁-C₄-perfluoroalkyl;
(d) aryl or —(C₁-C₄-alkyl)-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)O—, —C(O)—N(R^{a})—, —N(R^{a})—C(O)—, —N(R^{a})—C(O)—N(R^{a})—, —C(O)—, —CH(OH)—, —C═C—, or —C≡C—;
(e) —C(═O)(R^{a}), —CONR^{a}R^{b}, COO—(C₁-C₄)alkyl, —SO₂R^{a}, —SO₂N(R^{a})(R^{b});
R³ is
(a) H;
(b) —C₁-C₄-alkyl, —C₃-C₆-cycloalkyl or —C₁-C₄-alkyl-(C₃-C₆)-cycloalkyl, optionally substituted with one or more of the following substituents: F, CF₃, OH, O—(C₁-C₄)alkyl, S(O)₀₋₂—(C₁-C₄)alkyl, O—CONR^{a}R^{b}, NR^{a}R^{b}, N(R^{a}R^{b})CONR^{a}R^{b}, COO—(C₁-C₄)alkyl, COOH, CN, CONR^{a}R^{b}, SO₂NR^{a}R^{b}, N(R^{a}R^{b})SO₂NR^{a}R^{b}, —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl or piperazinyl;
(c) —C₀-C₄-alkyl-C₁-C₄-perfluoroalkyl;
(d) aryl or —(C₁-C₄-alkyl)-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a}) —C(O)—, —N(R^{a})—C(O)—N(R^{a})—, —C(O)—, —CH(OH)—, —C═C—, or —C≡C—;
(e) —O—C₁-C₄-alkyl, —O—C₀-C₄-alkyl-C₁-C₄-perfluoroalkyl, —O-aryl or —O(C₁-C₄-alkyl)-aryl;
(f) —C(═O)(R^{a}), —SO₂R^{a}, —SO₂N(R^{a})(R^{b}), CN, NR^{a}R^{b}, NO₂, F, Cl, Br, I, OH, OCONR^{a}R^{b}, O(C₁-C₄-alkyl)CONR^{a}R^{b}, —OSO₂NR^{a}R^{b}, COOR^{a}, or CONR^{a}R^{b};
R⁴ and R⁵ each independently is:
(a) H;
(b) —C₁-C₆-alkyl, —C₁-C₆-alkenyl, —C₂-C₆-alkynyl or —C₃-C₆-cycloalkyl, any of which is optionally substituted with one or more of the following substituents: F, CF₃, —O—(C₁-C₄)alkyl, CN, —N(R^{a})(R^{b}), —N(R^{a})CO—(C₁-C₄)alkyl, COOR^{b}, CON(R^{a})(R^{a}) or phenyl;
(c) —O—C₀-C₆-alkyl, —O-aryl, or —O—C₁-C₄-alkyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-N—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C—;
(d) —C₀-C₄-alkyl-C₁-C₄-perfluoroalkyl, or —O—C₀-C₄-alkyl-C₁-C₄-perfluoroalkyl; or
(e) CN, NH₂, NO₂, F, Cl, Br, I, OH, OCON(R^{a})(R^{b})O(C₁-C₄-alkyl)CONR^{a}R^{b}, —OSO₂N(R^{a})(R^{b}), COOR^{b}, CON(R^{a})(R^{b}), or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(—O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH)—, —C═C—, or —C≡C; and
R⁶, R⁷ and R⁸ each independently is:
(a) H;
(b) C₁-C₆-alkyl, C₂-C₄-alkenyl, C₃-C₄-alkynyl or C₃-C₆cycloalkyl, any of which is optionally substituted with one or more of the following substituents: F, CF₃, OH, O—(C₁-C₄)alkyl, OCON(R^{a})(R^{b}), NR^{a}R^{b}, COOR^{a}, CN, CONR^{a}R^{b}, N(R^{a})CONR^{a}R^{b}, N(R^{a})SO₂NR^{a}R^{b}, SO₂NR^{a}R^{b}, S(O)₀₋₂(C₁-C₄-alkyl), —C(═NH)NH₂, tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(c) —O—C₁-C₆-alkyl, —O—C₃-C₆-cycloalkyl, —S—C₁-C₆-alkyl or —S—C₃-C₆-cycloalkyl, any of which is optionally substituted with one or more of the following substituents: F, CF₃, OH, O—(C₁-C₄)alkyl, NH₂, NH(C₁-C₄-alkyl), N(C₁-C₄-alkyl)₂, COOH, CN, CONH₂, CONH(C₁-C₄-alkyl), CONH(C₁-C₄-alkyl)₂, SO₂NH₂, SO₂NH(C₁-C₄-alkyl), tetrazolyl, triazolyl, imidazolyl, oxazolyl, oxadiazolyl, isooxazolyl, thiazolyl, furyl, thienyl, pyrazolyl, pyrrolyl, pyridyl, pyrimidinyl, pyrazinyl, phenyl, piperidinyl, morpholinyl, pyrrolidinyl, or piperazinyl;
(d) —C₀-C₄-alkyl-C₁-C₄-perfluoroalkyl, or —O—C₀-C₄-alkyl-C₁-C₄-perfluoroalkyl;
(e) —O-aryl, or —O—C₁-C₄-allyl-aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, L ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)-CO—N(R^{a})(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a}) —C(O)—, —N(R^{a})—C(O)—, —CH(OH)—, —C≡C—, or —C≡C; (f) CN, N(R^{a})(R^{b}), NO₂, F, Cl, Br, I, —OR^{a}, —SR^{a}, —OCON(R^{a})(R^{b}), —OSO₂N(R^{a})(R^{b}), COOR^{b}, CON(R^{a})(R^{b}), —N(R^{a})CON(R^{a})(R^{b}), —N(R^{a})SO₂N(R^{a})(R^{b}), —C(OR^{b})R^{a}, —C(OR^{a})CF₃, —C(NHR^{a})CF₃, —C(═O)R^{a}, C(═O)CF₃, —SOCH₃, —SO₂CH₃, —NHSO₂(C₁₋₆-alkyl), —NHSO₂-aryl, SO₂N(R^{a})(R^{b}), —CH₂OSO₂N(R^{a})(R^{b}), SO₂N(R^{b})—OR^{a}, —C(═NH)NH₂, —CRₐ═N—ORₐ, CH═CH or aryl, wherein aryl is phenyl, pyridyl, pyrimidinyl, furyl, thienyl, pyrrolyl, triazolyl, pyrazolyl, thiazolyl, isoxazolyl, oxazolyl, or oxadiazolyl, any aryl of which is optionally substituted with 1-3 substituents selected from i) F, Cl, Br, I, ii) —CN, iii) —NO₂, iv) —C(═O)(R^{a}), v) —OR^{a}, vi) —NR^{a}R^{b}, vii) —C₀₋₄alkyl-CO—OR^{a}, viii) —(C₀₋₄alkyl)-NH—CO—OR^{a}, ix) —(C₀₋₄alkyl)—CO—N(W)(R^{b}), x) —S(O)₀₋₂R^{a}, xi) —SO₂N(R^{a})(R^{b}), xii) —NR^{a}SO₂R^{a}, xiii) —C₁₋₁₀alkyl, and xiv) —C₁₋₁₀alkyl, wherein one or more of the alkyl carbons can be replaced by a —NR^{a}—, —O—, —S(O)₁₋₂—, —O—C(O)—, —C(O)—O—, —C(O)—N(R^{a})—, —N(R^{a})—C(O)—, —N(R^{a})—C(O)—N(R^{a})—, —C(O)—, —CH(OH), —C═C—, or —C≡C; or when R⁶ and R⁷ are present on adjacent carbon atoms, R⁶ and R⁷, together with the benzene ring to which they are attached, may form a bicyclic aromatic ring selected from naphthyl, indolyl, quinolinyl, isoquinolinyl, quinoxalinyl. benzofuryl, benzothienyl, benzoxazolyl, benzothiazolyl, and benzimidazolyl, any aromatic ring of which is optionally substituted with 1-4 independent substituents selected from i) halogen, ii) —CN, iii) —NO₂, iv) —CHO, v) —O—C₁₋₄alkyl, vi) —N(C₀₋₄alkyl)(C₀₋₄alkyl), vii) —C₀₋₄alkyl-CO—O(C₀₋₄alkyl), viii) —(C₀₋₄alkyl)-NH—CO—O(C₀₋₄alkyl), ix) —(C₀₋₄alkyl)-CO—N(C₀₋₄alkyl)(C₀₋₄alkyl), x) —S(C₀₋₄alkyl), xi) —S(O)(C₁₋₄alkyl), xii) —SO₂(C₀₋₄alkyl), xiii) —SO₂N(C₀₋₄alkyl)(C₀₋₄alkyl), xiv) —NHSO₂(C₀₋₄alkyl)(C₀₋₄alkyl), xv) —C₁₋₁₀alkyl and xvi) —C₁₋₁₀alkyl in which one or more of the carbons can be replaced by a —N(C₀₋₆alkyl)-, —O—, —S(O)₁₋₂—, —O—C(O)—, —C(O)—O—, —C(O)—N(C₀₋₆alkyl)-, —N(C₀₋₆alkyl)-C(O)—, —N(C₀₋₆alkyl)-C(O)—N(C₀₋₆alkyl)-, —C(O)—, —CH(OH), —C═C—, or —C≡C—.

2. A compound according to claim 1, or a pharmaceutically acceptable salt thereof,
wherein
HET is

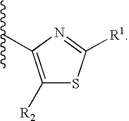

3. A compound according to claim 1, or a pharmaceutically acceptable salt thereof,
wherein
HET is

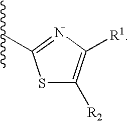

4. A compound according to claim 1, or a pharmaceutically acceptable salt thereof,
wherein
HET is

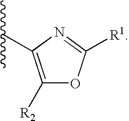

5. A compound according to claim 1, or a pharmaceutically acceptable salt thereof,
wherein
HET is

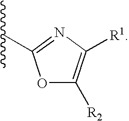

6. A compound according to claim 1, or a pharmaceutically acceptable salt thereof,
wherein
HET is

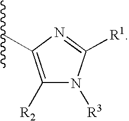

7. A compound according to claim 1, or a pharmaceutically acceptable salt thereof,
wherein
HET is

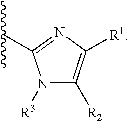

8. A compound according to claim 1, or a pharmaceutically acceptable salt thereof,
wherein
R⁶ is other than H and is attached at the ortho position.

9. A compound represented by

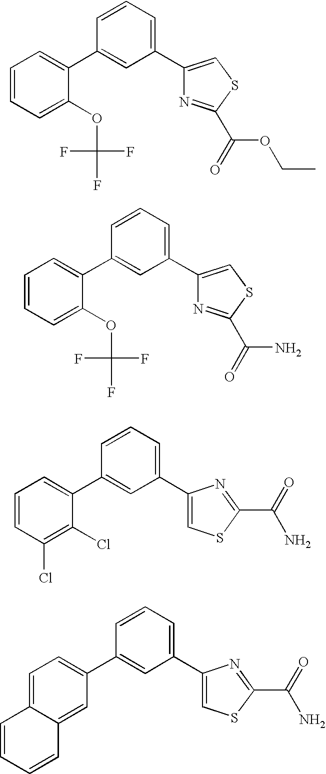

10. A compound according to claim 1 represented by

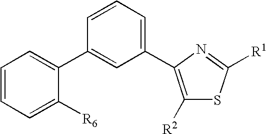

| R⁶ | R² | R¹ |
|---|---|---|
| Cl | H | H |
| Cl | H | COOEt |
| Cl | H | CONH₂ |
| Cl | H | CONH-tBu |
| Cl | H | 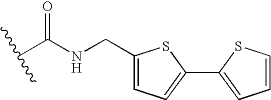 |
| Cl | H | NH₂ |
| CF₃ | H | COOEt |
| CF₃ | H | CONH₂ |
| CF₃ | H | H |
| CF₃ | H | NH₂ |
| OCF₃ | H | CH₃ |
| OCF₃ | H | H |
| OCF₃ | H | NH₂ |
| OCF₃ | H | CONMe₂ |
| OCF₃ | Cl | CH₃ |
| OCF₃ | H | NHSO₂CH₃ |
| OCF₃ | H | CH₂OH |
| O—Ph | H | CONH₂ |
| CF₃ | H | NHCONH-iPr |
| OCF₃ | H | NHCONH-iPr |
| OCF₃ | H | NHCOCH₃ |
| CF₃ | H | NHCOCH₃ |
| OCF₃ | H | CH₂COOEt |
| OCF₃ | H | CH₂CN |
| OCF₃ | H | CH₂CONH₂ |
| CF₃ | H | CH₂CONH₂ |
| OCF₃ | H | NHCONMe₂ |
| OCF₃ | H | 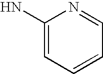 |
| OCF₃ | H | 2-Pyrimidyl |
| OCF₃ | H | 2-Pyridyl |
| OCF₃ | H | 2-Oxazolyl |
| OCF₃ | H | 2-Imidazolyl |
| OCF₃ | H | 2-Pyrazolyl |
| OCF₃ | H | 2-(1-Methyl)- |
| | | imidazolyl |
| OCF₃ | H | 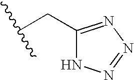 |
| OCF₃ | H | 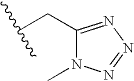 |
| OCF₃ | H | 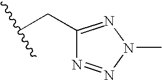 |

11. A compound represented by

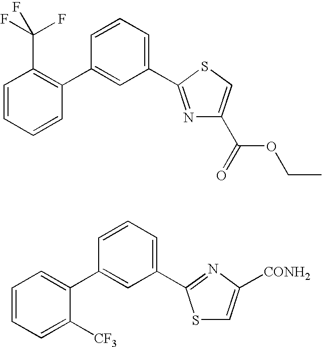

12. A compound according to claim 1 represented by

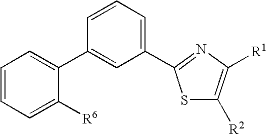

| R₆ | R₂ | R₁ |
|---|---|---|
| CF₃ | H | H |
| CF₃ | H | COOEt |
| CF₃ | H | CONH₂ |
| CF₃ | H | CONHCH₃ |
| CF₃ | COOEt | CH₃ |
| CF₃ | CONH₂ | CH₃ |
| OCF₃ | H | H |
| OCF₃ | H | COOCH₃ |
| OCF₃ | H | CONH₂ |
| OCF₃ | H | COOH |
| OCF₃ | H | CH₂OH |
| OCF₃ | H | CONH(CH₂)₃OH |
| O—Ph | H | CONH₂ |

13. A compound represented by

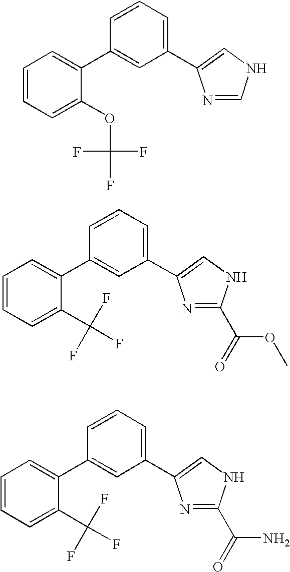

14. A compound according to claim 1 represented by

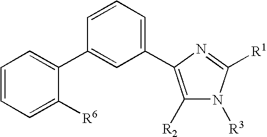

| R⁶ | R³ | R² | R¹ |
|---|---|---|---|
| Cl | H | H | H |
| Cl | H | H | COOCH₃ |
| Cl | H | H | CONH₂ |
| CF₃ | H | H | H |
| CF₃ | H | H | COOH |
| CF₃ | CH₃ | H | CONH₂ |
| CF₃ | H | Cl | COOCH₃ |
| CF₃ | H | Cl | CONH₂ |
| CF₃ | H | Cl | Cl |
| OCF₃ | H | H | H |
| OCF₃ | H | H | COOCH₃ |
| OCF₃ | H | H | CONH₂ |
| OCF₃ | H | H | 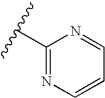 |

15. A compound represented by

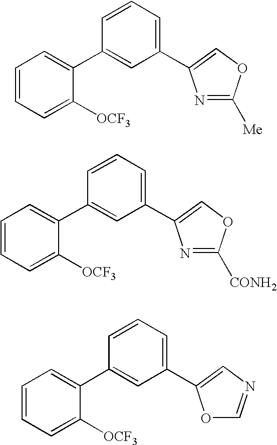

16. A compound according to claim 1 represented by

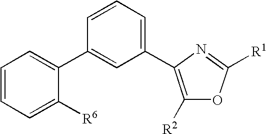

| R⁶ | R² | R¹ |
|---|---|---|
| Cl | H | CH₃ |
| Cl | H | NH₂ |
| CF₃ | H | CH₃ |
| OCF₃ | H | NH₂ |
| OCF₃ | H | CH═CH₂ |
| OCF₃ | H | COOCH₃ |
| OCF₃ | H | CONH₂ |
| OCF₃ | H | CH₃ |
| CF₃ | H | NH₂ |
| CF₃ | H | H |
| OCF₃ | H | H |
| Cl | H | COOH |
| CF₃ | H | CONH₂ |
| OCF₃ | H | COOCH₂CH₃ |

17. A pharmaceutical composition comprising a therapeutically effective amount of the compound according to claim 1, or a pharmaceutically acceptable salt thereof, and a pharmaceutically acceptable carrier.

18. The pharmaceutical composition according to claim 17, further comprising a second therapeutic agent selected from the group consisting of: i) opiate agonists, ii) opiate antagonists, iii) calcium channel antagonists, iv) 5HT receptor agonists, v) 5HT receptor antagonists vi) sodium channel antagonists, vii) NMDA receptor agonists, viii) NMDA receptor antagonists, ix) COX-2 selective inhibitors, x) NK1 antagonists, xi) non-steroidal anti-inflammatory drugs, xii) selective serotonin reuptake inhibitors, xiii) selective serotonin and norepinephrine reuptake inhibitors, xiv) tricyclic antidepressant drugs, xv) norepinephrine modulators, xvi) lithium, xvii) valproate xviii) neurontin, and xix) antiviral agents.

19. A method of treatment or prevention of pain comprising the step of administering to a patient in need thereof a therapeutically effective amount, or a prophylactically effective amount, of the compound according to claim 1, or a pharmaceutically acceptable salt thereof.

20. A method of treatment of chronic, visceral, inflammatory and neuropathic pain syndromes comprising the step of administering to a patient in need thereof a therapeutically effective amount, or a prophylactically effective amount, of the compound according to claim 1, or a pharmaceutically acceptable salt thereof.

21. A method of treatment of pain resulting from, or associated with, traumatic nerve injury, nerve compression or entrapment, postherpetic neuralgia, trigeminal neuralgia, diabetic neuropathy, cancer and chemotherapy, comprising the step of administering to a patient in need thereof a therapeutically effective amount, or a prophylactically effective amount, of the compound according to claim 1, or a pharmaceutically acceptable salt thereof.

22. A method of treatment of chronic lower back pain comprising the step of administering to a patient in need thereof a therapeutically effective amount, or a prophylactically effective amount, of the compound according to claim 1, or a pharmaceutically acceptable salt thereof.

23. A method of treatment of phantom limb pain comprising the step of administering to a patient in need thereof a therapeutically effective amount, or a prophylactically effective amount, of the compound according to claim 1, or a pharmaceutically acceptable salt thereof.

24. A method of treatment of HIV- and HIV treatment-induced neuropathy, chronic pelvic pain, neuroma pain, complex regional pain syndrome, chronic arthritic pain and related neuralgias comprising the step of administering to a patient in need thereof a therapeutically effective amount, or a prophylactically effective amount, of the compound according to claim 1, or a pharmaceutically acceptable salt thereof.

25. A method of administering local anesthesia comprising the step of administering to a patient in need thereof a therapeutically effective amount, or a prophylactically effective amount, of the compound according to claim 1, or a pharmaceutically acceptable salt thereof.

26. A method of treatment of irritable bowel syndrome and Crohn's disease comprising the step of administering to a patient in need thereof a therapeutically effective amount, or a prophylactically effective amount, of the compound according to claim 1, or a pharmaceutically acceptable salt thereof.

27. A method of treatment of epilepsy and partial and generalized tonic seizures comprising the step of administering to a patient in need thereof a therapeutically effective amount, or a prophylactically effective amount, of the compound according to claim 1, or a pharmaceutically acceptable salt thereof.

28. A method for neuroprotection under ischaemic conditions caused by stroke or neural trauma comprising the step of administering to a patient in need thereof a therapeutically effective amount, or a prophylactically effective amount, of the compound according to claim 1, or a pharmaceutically acceptable salt thereof.

29. A method of treatment of multiple sclerosis comprising the step of administering to a patient in need thereof a therapeutically effective amount, or a prophylactically effective amount, of the compound according to claim 1, or a pharmaceutically acceptable salt thereof.

30. A method of treatment of bipolar disorder comprising the step of administering to a patient in need thereof a therapeutically effective amount, or a prophylactically effective amount, of the compound according to claim 1, or a pharmaceutically acceptable salt thereof.

31. A method of treatment of tachy-arrhythmias comprising the step of administering to a patient in need thereof a therapeutically effective amount, or a prophylactically effective amount, of the compound according to claim 1, or a pharmaceutically acceptable salt thereof.
